(19) United States
(12) Patent Application Publication
(10) Pub. No.: US 2002/0048572 A1
(43) Pub. Date: Apr. 25, 2002

(54) Treatment of hypertriglyceridemia and other conditions using LXR modulators

(76) Inventors: Bei Shan, Redwood City, CA (US); Joshua Schultz, Half Moon Bay, CA (US); Hua Tu, San Bruno, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/848,990

(22) Filed: May 3, 2001

Related U.S. Application Data
(60) Provisional application No. 60/201,601, filed on May 3, 2000.

(51) Int. Cl. A61K 38/43; A61K 31/58

(52) U.S. Cl. 424/94.1; 514/172

(57) ABSTRACT

This invention provides methods for identifying compounds that are suitable for use in modulating fatty acid and triglyceride biosynthesis, and thus treating conditions such as hypertriglyceridemia and lipodystrophy, among others. Provided are in vitro assays by which one can conduct prescreening to identify candidate therapeutic agents that are suitable for further testing, as well as assays for identifying agents that are useful for administration for treating conditions associated with abnormalities in fatty acid and triglyceride biosynthesis.

### CROSS-REFERENCE TO RELATED APPLICATION

This application claims the benefit of U.S. Provisional Patent Application Serial No. 60/201,601, filed May 3, 2000, which is incorporated herein by reference in its entirety for all purposes.

### BACKGROUND OF THE INVENTION

1. Field of the Invention

This invention pertains to the field of the study and treatment of conditions that involve abnormalities in fatty acid and triglyceride biosynthesis.

2. Background

Hypertriglyceridemia is a major risk factor for coronary heart disease. For each mmol/L increase in triglycerides (i.e., 88.5 mg/dL) the risk of coronary heart disease increases by 37% in women and 14% in men (Hokanson and Austin, J. Cardiovasc. Risk 3:231 (1996)). A recent study found that the risk of major cardiovascular events was reduced in patients with coronary disease by a treatment that lowered triglycerides and raised HDL cholesterol levels, but did not lower LDL cholesterol levels (Rubins et al, New Engl. J. Med. 341:410-418 (1999)).

High triglyceride levels can also result in conditions such as pancreatitis. Lipodystrophy, a disorder of lipid metabolism, has clinical symptoms that include hypertriglyceridemia, as well as loss of body fat, severe diabetes, voracious appetite and organomegaly of the liver, spleen, pancreas and kidney. Other symptoms include insulin resistance and hyperglycemia (see, e.g., Harrison's Principles of Internal Medicine, Wilson et al., Eds., 12^{th} Ed., pp. 1883-1885 (McGraw-Hill, Inc., New York). Early death often results for patients having generalized lipodystrophy, in which essentially all body fat is lost. Partial lipodystrophy involves more limited fat atrophy. Both congenitally transmitted and acquired forms of lipodystrophy are known. Lipodystrophy is also commonly observed in HIV positive individuals who are undergoing treatment with protease inhibitors. For example, protease inhibitors can cause “protease belly,” in which the belly of a patient becomes large and protruding. As is the case for congenitally transmitted lipodystrophy, affected patients often have elevated levels of triglycerides, liver enzymes, and blood glucose. Diabetes has also been observed in such patients.

In addition to the causal factors such as protease inhibitors and genetic lipodystrophy, hypertriglyceridemia can result from several causes, including genetic disorders such as lipoprotein lipase abnormalities, abnormalities in apolipoprotien CII or CIII, Apo E abnormalities, familial hypertriglyceridemia, and familial combined hyperlipidemia. Metabolic disorders, including diabetes mellitus, hypothyroidism, paraproteinemia, and nephrotic syndrome/renal failure, can also result in hypertriglyceridemia. Hypertriglyceridemia can also result from medications, such as bile acid resins, retinoids, beta blockers, steroids, and estrogenic compounds (Michael Miller, Hospital Practice, “Giving triglycerides their due,” The McGraw-Hill Companies, 2000 (http://www.hosppract.com/issues/1999/0901/miller.htm).

Transgenic mice that constitutively express a gene that encodes sterol regulatory element-binding protein (SREBP)-1 (also known as adipocyte determination and differentiation factor-1, ADD-1) exhibit a phenotype that is markedly similar to that observed in congenital or acquired lipodystrophy (Shimomura et al., Genes and Devel. 12:3182-3194 (1998); see also, McKnight, Genes and Devel. 12:3145-3148 (1998)). These mice exhibited differentiation of adipose tissue, as well as reduced levels of mRNAs that encode the adipocyte differentiation markers C/EBPα, PPARγ, adipsin, leptin, and UCP1. Insulin levels were elevated 60-fold, and the mice exhibited marked insulin resistance. The transgenic mice also had diabetes mellitus and elevated blood glucose, fatty livers, and elevated plasma triglyceride levels.

The SREBPs are transcription factors that are involved in regulation of genes involved in fatty acid and cholesterol biosynthesis and thus control the lipid composition of animal cells (FIG. 1). Three isoforms of mature SREBP polypeptide are known, SREBP-1a, SREBP-1c and SREBP-2. SREBP-1a and -1c are mRNA splicing variants that are encoded by a single gene (Yokoyama et al., Cell 75:187-197 (1993); Hua et al., Genomics 25:667-673 (1995)). SREBP-1a and -1c preferentially regulate the fatty acid biosynthesis pathway, while SREBP-2 is involved in regulation of cholesterol biosynthesis (Horton et al., J. Clin. Invest. 101:2331-2339 (1998); Pai et al., J. Biol. Chem. 273:26138-26148 (1998)). These transcription factors are bound to membranes of the endoplasmic reticulum (ER) and nuclear envelope, and are released by sterol-regulated proteolysis. SREBP release is initiated by Site-b 1 protease (SIP), which cleaves SREBPs in the ER luminal loop between two membrane spanning regions (see, for example, Sakai et al., Cell 85:1037-1046 (1996); Duncan et al., J. Biol. Chem. 272:12778-12785 (1997); copending Application Ser. No. 09/360,237, entitled “cDNA Cloning of Site-1 Protease for SREBPs”), and copending Provisional Application No. 60/159,236, filed Oct. 13, 1999, entitled “Modulators of SREBP1 Processing.”

Knockout mice were recently constructed in which expression of the nuclear receptor LXRα was eliminated (Peet et al., Cell 93:693-704 (1998)). The LXRs were first identified as orphan members of the nuclear receptor superfamily whose ligands and functions were unknown. Two LXR proteins α and β are known to exist in mammals. The expression of LXRα is restricted, with the highest levels being found in the liver, and lower levels found in the kidney, intestine, spleen, and adrenals (Willy et al., Genes Dev. 9(9):1033-45 (1995)). LXRβ is rather ubiquitous, being found in nearly all tissues examined. Recent studies on the LXRs indicate that they are activated by certain naturally occurring, oxidized derivatives of cholesterol, including 22(R)-hydroxycholesterol, 24(S)-hydroxycholesterol, and 24,25(S)-epoxycholesterol (Lehmann et al, J. Biol. Chem. 272(6):3137-3140 (1997)). When fed a low-cholesterol diet, LXRα knockout mice exhibited reduced levels of genes involved in fatty acid synthesis, such as stearoyl CoA desaturase-1, fatty acid synthase, and SREBP-1.

No effective treatment exists for hypertriglyceridemia. In the case of lipodystrophy, a transgenic mouse model system in which targeted expression of diphtheria toxin A destroyed adipose tissue (Burant et al., J. Clin. Invest. 100:2900-2908 (1997)) was used to demonstrate that oral treatment of mice with troglitazone, which activates PPARγ, resulted in improvements in hyperglycemia and insulin resistance (Spiegelman and Flier, Cell 87:377-389 (1996)). However, this treatment may not be effective for treating other symptoms of lipodystrophy, such as hypertriglyceridemia. Although the lipodystrophic phenotype of the SREBP-1c-overexpressing transgenic mice raises the possibility that SREBP-1c is involved in lipodystrophy, the mechanism by which SREBP-1 expression is regulated is not yet understood. Nor are ways to reduce SREBP-1 expression. Thus, a need exists for an enhanced understanding of the mechanism of lipodystrophy and hypertriglyceridemia. Particularly important is an understanding of how SREBP-1 expression is regulated, and also for ways to modulate the expression of SREBP-1. The present invention fulfills these and other needs.

### BRIEF SUMMARY OF THE INVENTION

The present invention provides methods for modulating expression of a mammalian SREBP-1 gene. SREBP-1 is involved in regulating expression of genes that encode enzymes involved in fatty acid and triglyceride metabolism. The methods involve administering a modulator compound that promotes or inhibits LXRα-mediated expression of the SREBP-1 gene (e.g., SREBP-1a and/or SREBP-1c) to a cell that comprises an SREBP-1 gene and an LXRα polypeptide.

In another embodiment, the invention provides methods of modulating triglyceride levels in a mammal. These methods involve administering to the mammal an effective amount of a modulator compound that inhibits LXRα-mediated expression of an SREBP-1 gene in cells of the mammal.

Also provided by the invention are methods of prescreening to identify a candidate therapeutic agent that modulates SREBP-1 expression in a mammal. The methods involve:

providing a reaction mixture which comprises:

a polypeptide that comprises an LXRα ligand binding domain

(LBD);

a ligand for LXRα; and

a test compound; and

determining whether the amount of LXRα ligand that binds to the LBD is increased or decreased in the presence of the test compound relative to the amount of ligand that binds to the LBD in the absence of the test compound. A test compound that causes an increase or decrease in the amount of LXRα ligand binding to the LBD is a candidate therapeutic agent for modulation of SREBP-1 expression in a mammal.

### BRIEF DESCRIPTION OF THE DRAWINGS

FIG. 1 shows a schematic diagram of the cholesterol, fatty acid and phospholipid biosynthesis pathways. SREBP-1 stimulates expression of the fatty acid biosynthesis, while SREBP-2 regulates the cholesterol and phospholipid pathways.

FIG. 2 shows data which demonstrate that the LXRα ligand 24,25-epoxycholesterol activates expression of SREBP-1 mRNA, both in the presence and absence of cyclohexamide.

FIG. 3 presents an autoradiogram which demonstrates that 24,25-epoxycholesterol activates SREBP-1 expression in different cell lines, including HepG2, 293, and CaCO₂ cells

FIG. 4 shows the results of an experiment in which the effect on expression of a luciferase reporter gene which is under the control of promoters from SREBP-1a, SREBP-1, 3X-LXRE-TK, or 3X-CPFRE-TK was determined in the presence of a plasmid that encodes LXR alone, LXR and RXR, or the expression vector with no insert (pc). Expression of SREBP-1 and the positive control 3X-LXRE was stimulated in cells that contain both LXR and RXR.

FIG. 5 shows the location of putative LXR response elements in the human SREBP-1a and SREBP-1c upstream regions. Also shown is the nucleotide sequence of the region upstream of exon 1c, which region includes the promoter for human SREBP-1c. Putative LXRα response elements are underlined.

FIG. 6. Biochemical characterization of LXR ligands T0314407 and T0901317. (A) Chemical structure of T0314407, T0901317, and radiolabeled T0314407. (B) Effect of different LXRα agonists on recruitment of an LXXLL-containing peptide to the LXRα ligand-binding domain. (C) Scintillation proximity assay (SPA) data showing the competitive binding curves of (³H)-TO314407 to LXRα protein and the ability of unlabeled LXR ligands to displace (³H)-T0314407

FIG. 7. (A) Dose responses to LXR ligands in an HEK293 transient transfection assay using a wild-type LXRα expression plasmid and a luciferase reporter gene containing two copies of an LXR response element. DMSO treatments were used to derive the basal level of activation. (B) Specificity of LXR activation by LXR ligands in a transient transfection assay. HEK293 cells were cotransfected with a luciferase reporter gene containing four copies of the Gal4 DNA-binding site and the various chimeric Gal4 (DNA-binding domain)-nuclear receptor (ligand-binding domain) proteins shown. Cells were treated with the indicated compounds at concentrations of 1 μM, 5 μM, and 10 μM for T0901317, T0314407, and 24,25-BC, respectively.

FIG. 8. Plasma lipid levels in animals treated with LXR agonist T0901317. The concentrations of (A) plasma triglycerides and (B) HDL-cholesterol for C57BL/6 mice (n=5, for each treatment group, M, males, F, females). (C) Separation of Golden Syrian hamster plasma by fast protein liquid chromatography (FPLC) in combination with analysis of the triglyceride content across PPLC fractions. Lipid data are reported as the mean ±SD for the number of determinations (animals) described in each experiment. Statistical significance (kybd) was defined as P<0.05 (ANOVA using SAS programming statistical test for controls and LXR agonist-treated animals).

FIG. 9. Northern blot analysis. Total RNA was isolated from the tissues indicated from mice (A,B,C) and hamsters (D). The numerical data shown with each figure represents the fold increase (+) or decrease (−) of expression relative to corresponding vehicle-treated controls. (E) HepG2 cells were incubated in culture media with or without cycloheximide (cyclohex).

FIG. 10. Agonist T0901317-mediated increases in hepatic and plasma triglyceride levels and lipogenic mRNA requires LXRs. Analysis of the lipid content of livers and plasma from vehicle- and T0901317-created wild-type and LXRα/β-/- mice (n=5 for each of the four groups shown, treatment for 7 d). Pooled plasma (50 μL from each of five mice per group) was fractionated and lipids analyzed as described. Shown are the concentrations for (A) FPLC triglyceride, (B) FPLC phospholipid, and (C) hepatic triglyceride. (D) Northern blot analysis of hepatic SREEP-I, SREBP-2, SCD-1, FAS, and ACC mRNA levels.

FIG. 11. The sequence of PCR primers used for amplifying mouse cDNA probes.

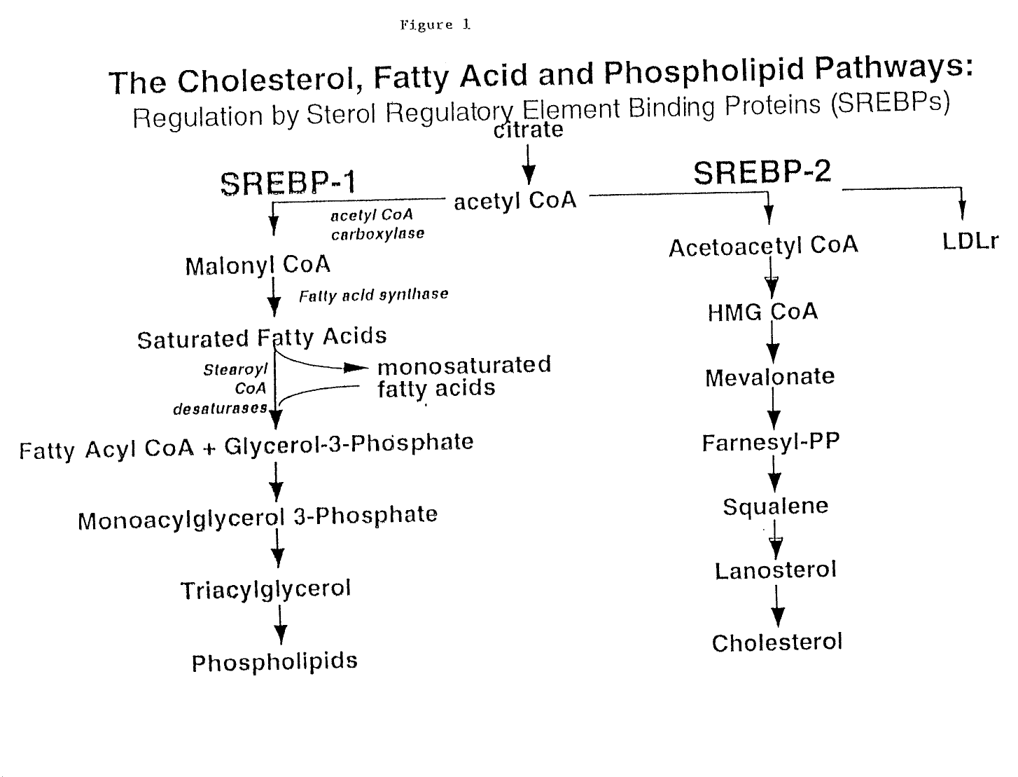

### DETAILED DESCRIPTION OF THE INVENTION AND PREFERRED EMBODIMENTS

### Definitions

The term “isolated” refers to material that is substantially or essentially free from components which normally accompany the enzyme as found in its native state. Thus, the polypeptides of the invention do not include materials normally associated with their in situ environment. Typically, isolated proteins of the invention are at least about 80% pure, usually at least about 90%, and preferably at least about 95% pure as measured by band intensity on a silver stained gel or other method for determining purity. Protein purity or homogeneity can be indicated by a number of means well known in the art, such as polyacrylamide gel electrophoresis of a protein sample, followed by visualization upon staining. For certain purposes, high resolution will be needed and HPLC or a similar means for purification utilized.

The term “recombinant” when used with reference to a cell, or nucleic acid, or vector, indicates that the cell, or nucleic acid, or vector, has been modified by the introduction of a heterologous nucleic acid or the alteration of a native nucleic acid, or that the cell is derived from a cell so modified. Thus, for example, recombinant cells express genes that are not found within the native (nonrecombinant) form of the cell or express native genes that are otherwise abnormally expressed, under expressed or not expressed at all.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual alignment and inspection.

The phrase “substantially identical,” in the context of two nucleic acids or polypeptides, refers to two or more sequences or subsequences that have at least 75%, preferably 85%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions and/or untranslated regions.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA. The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na⁺, typically about 0.01 to 1.0 M Na⁺ concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide.

The terms “specifically binds” or “selectively binds”, refers to a binding reaction which is determinative of the presence of the protein or other component in the presence of a heterogeneous population of proteins and other biologics. Thus, under designated assay conditions, the specified ligands bind to a particular protein (e.g., LXRα) and do not bind in a significant amount to other proteins present in the sample. Specific binding to an antibody under such conditions may require an antibody that is selected for its specificity for a particular protein. For example, antibodies raised to the LXRα polypeptides (or subsequences thereof) or to the polypeptides partially encoded by the LXRα polynucleotide sequences can be selected to obtain antibodies specifically immunoreactive with the full length proteins and not with other proteins, except perhaps to polymorphic variants. A variety of assay formats can be used to select antibodies and other molecules that specifically bind to a particular protein such as LXRα. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity. Typically, a specific or selective reaction will be at least twice background signal or noise and more typically more than 10 to 100 times background.

### DESCRIPTION OF THE PREFERRED EMBODIMENTS

The present invention provides methods and kits for reducing expression of mammalian SREBP-1 genes. The invention is based upon the discovery that antagonists of the nuclear receptor LXRα can inhibit SREBP-1 expression in mammalian cells. Abnormal expression of SREBP-1 is involved in conditions such as hypertriglyceridemia and lipodystrophy. Accordingly, the invention also provides methods and kits for ameliorating conditions such as lipodystrophy, hypertriglyceridemia, hyperglyceremia and diabetes. Also provided are methods for screening to identify candidate therapeutic agents that are suitable for further testing or screening for ability to act as an LXRα antagonist (or an LXRα agonist).

The methods and kits of the invention are useful not only for therapeutic and prophylactic treatment of conditions that are mediated by SREBP-1 overexpression, but also are useful for studies of the mechanisms of fatty acid homeostasis, and the causes and mechanisms of lipodystrophy.

A. Assays for Identifying Compounds that Modulate SREBP-1 Expression

The invention provides methods for identifying compounds that are suitable for further testing as candidate therapeutic agents for treatment of hypertriglyceridemia, lipodystrophy, and other conditions that are associated with fatty acid and triglyceride biosynthesis and metabolism. Compounds that exhibit the desired activity in the in vitro assays can be used for further studies of the regulation of pathways involved in fatty acid and triglyceride biosynthesis, or can be subjected for further testing to identify those that are suitable for use to treat hypertriglyceridemia and other conditions, either in their present form or after further derivatization and screening.

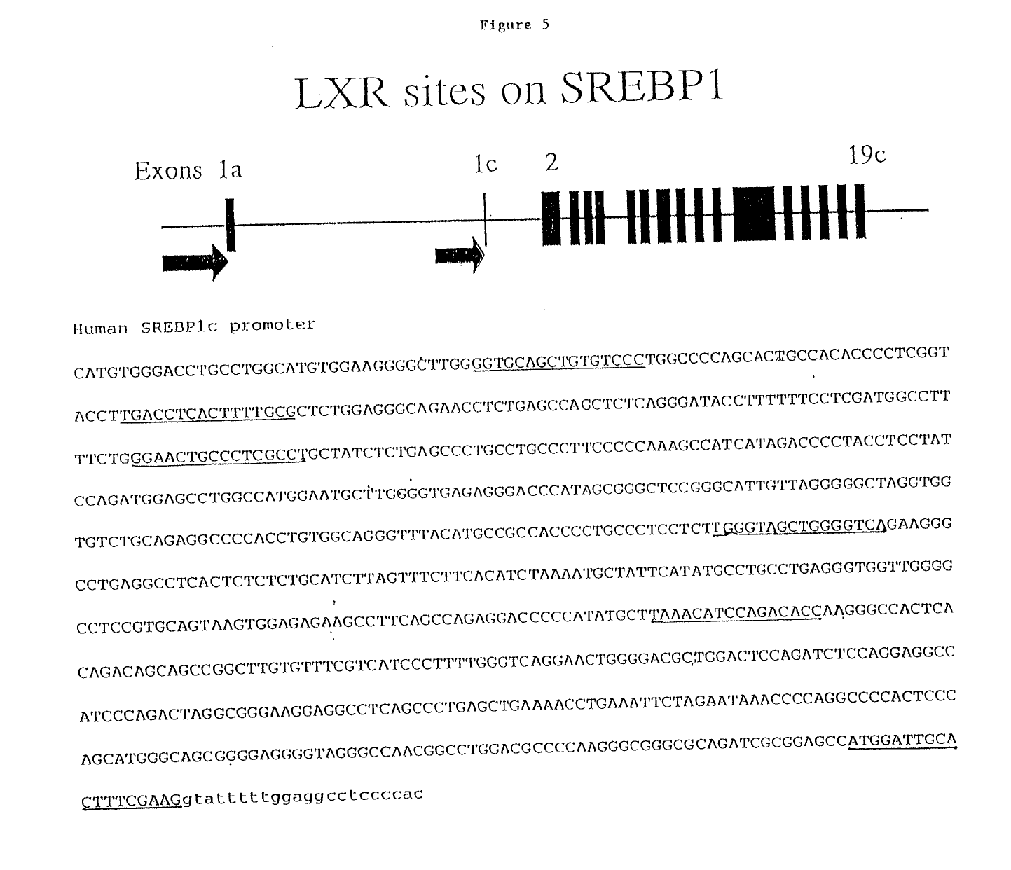

For example, the invention provides screening assays for identifying compounds that can modulate SREBP-1 (e.g., SREBP-1a and/or SREBP-1c) expression in mammalian cells. These compounds can function by, for example, altering the interaction between LXRα and ligands of LXRα (e.g., oxysterols, RXR, corepressors and/or coactivators, and the like) and/or between LXRα and its response elements. Of particular interest is the reduction of SREBP-1c expression, which is inhibited by binding of LXRα to the LXR response elements that are found upstream of exon 1c of the SREBP-1 gene (FIG. 5). A compound that inhibits the cis-activating activity of LXRα can decrease the expression of SREBP-1c and/or SREBP-1a, thus reducing the expression of enzymes that are involved in fatty acid and triglyceride biosynthesis. Expression of other genes that are involved in lipodystrophy is also modulated by the reduction in SREBP-1c and/or SREBP-1a levels. Thus, compounds that are identified using the screening methods of the invention find use in studies of gene regulation, and also find therapeutic use in situations in which it is desirable to decrease triglyceride and fatty acid biosynthesis. Other uses will also be apparent to those of ordinary skill in the art.

The assays of the invention are amenable to screening of large chemical libraries by automating the assay steps and providing compounds from any convenient source to assays, which are typically run in parallel (e.g., in microtiter formats on microtiter plates in robotic assays). Essentially any chemical compound can be used as a potential LXRα activity modulator in the assays of the invention. In some embodiments, compounds that can be dissolved in aqueous or organic (especially DMSO-based) solutions are used. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial library containing a large number of potential therapeutic compounds (potential modulator compounds). Such “combinatorial chemical libraries” are then screened in one or more assays, as described herein, to identify those library members (particular chemical species or subclasses) that display a desired characteristic activity (e.g., inhibit the interaction between LXRα and an LXRα ligand). The compounds thus identified can serve as conventional “lead compounds” or “candidate therapeutic agents,” and can be derivatized for further testing to identify even more effective LXRα antagonist activity, or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al, J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

The invention provides both biochemical and cell-based assays for identifying compounds that can modulate LXRα-mediated regulation of fatty acid and triglyceride biosynthesis. Often, an initial assay is performed in vitro to identify compounds that are potential candidate therapeutic agents, after which such compounds are then tested in vivo by, for example, administering the compound to a test animal to determine whether cholesterol levels are affected.

1. Direct and Displacement Assays

One type of assay that can be used is a direct binding assay, which measures the amount of a compound that can bind to an LXRα polypeptide or to a polypeptide that has an LXRα ligand binding domain. These assays can be carried out using labeled candidate therapeutic agents which are then incubated with a polypeptide that has an LXRα ligand binding domain (e.g., a full-length LXRα polypeptide or a fusion protein). Labels include radioisotopes, immunochemicals, fluorophores, and the like. Those of skill in the art will recognize a variety of ways of separating the bound labeled candidate therapeutic agent from the free labeled candidate therapeutic agent. The affinity of the labeled candidate therapeutic agent for an LXRα polypeptide can be calculated using standard ligand binding methods.

Another type of assay that can be used to pre-screen candidate therapeutic agents involves testing the ability of a test compound to modulate binding of LXRα to a ligand for LXRα. These can be conducted, for example, as a direct binding assay with a labeled LXRα ligand in the presence of a candidate therapeutic agent. The assays involve placing the test compound into an assay mixture that includes at least a ligand binding domain of an LXRα polypeptide and a ligand for LXRα. The effect on binding of the LXRα ligand to LXRα is determined. A test compound that decreases the amount of labeled LXRα ligand that is bound to an LXRα polypeptide or a polypeptide that has an LXRα ligand binding domain, is of interest for future screening for its ability to reduce triglyceride and/or fatty acid levels in vivo.

Ligands that are suitable for use in the assays of the invention include, but are not limited to, oxysterols, such as 24,25-epoxycholesterol. Methods of identifying LXRα ligands are described below.

In presently preferred embodiments, an assay such as the fluorescence polarization assay or the fluorescence resonance energy transfer assay is employed to identify candidate therapeutic agents. These assays do not require the separation of bound and free labeled test compound. Fluorescence polarization (FP) or fluorescence anisotropy is a useful tool for the study of molecular interactions (see, e.g. http://www.panvera.com/tech/appguide/fpintro.html, Nov. 4, 1999). First, a molecule labeled with a fluorophore is excited with plane polarized light. If the fluorescent molecule stays stationary while in the excited state, light is emitted in the same polarized plane. If the excited fluorescently labeled molecule rotates out of the plane of the polarized light while in the excited state, light is emitted from the molecule in a different plane. For example, if vertical polarized light is used to excite the fluorophore, the emission spectra can be monitored in the vertical and horizontal planes. Fluorescence polarization is calculated as shown in the following Formula I:

Fluorescent polarization=P=(Int∥−Int^{⊥})/(Int∥+Int^{⊥}) I

In Formula I, Int ∥ is the intensity of the emission parallel to the excitation plane. Int^{⊥} is the intensity of the emission perpendicular to the excitation plane.

A small fluorescently labeled molecule, when free in solution, can emit depolarized light when excited with the proper wavelength of light. If, however, the molecule (e.g., a ligand) binds to a second molecule (e.g., a receptor) the fluorescently labeled molecule is more constrained so the light emitted is more polarized and the fluorescence polarization (FP) value is higher. Thus, a higher FP value indicates that the fluorescently labeled molecule is able to bind to the second molecule. A competition assay also can be performed using FP. If an unlabeled molecule is present in the solution, then it will compete for binding to the second molecule, e.g., the antibody and the FP value will be decreased. Thus, FP can be used in competitive assays.

Commercial assays exist to test the affinity of compounds for human estrogen receptor α and β using a fluorescently labeled estrogen compound (see, Panvera (Madison, Wis.) publications Lit.#'s L0069, L0082, L0084, L0095, L0072, L0085). Similarly, test compounds can be fluorescently labeled with a fluorophore that is active in a FP assay. For example, N-terminal amines of proteins, peptide, or peptide analogs can be labeled with fluorescein (Panvera, publications Lit. #L0057 and L0059) or a small fluorescent compound. Briefly, a fluorescein-C₆-succinimidyl ester can be conjugated to peptides or proteins. The fluorescein labeled peptide/protein can then be purified from the unreacted fluorescein-C6-succinimidyl ester using thin-layer chromatography or gel filtration chromatography. If the labeled test compound can bind to a polypeptide that has an LXRα ligand binding domain, the level of polarization is increased. The FP assay also can be used to assay the ability of a fluorescently labeled LXRα ligand to bind to an LXRα polypeptide.

Alternatively, a test compound can be screened for its ability to decrease the FP of a fluorescently labeled known LXRα ligand complexed with an LXRα polypeptide or a polypeptide comprising an LXRα ligand binding domain. Briefly, a known LXRα ligand is labeled with a fluorescent moiety. A test compound that decreases the FP value of the fluorescently labeled LXRα ligand and LXRα is displacing or inhibiting the ability of the fluorescently labeled LXRα ligand to bind to the ligand binding domain of LXRα.

Methods employing the technique of fluorescence resonance energy transfer (FRET) can be employed using the methods and compositions of the present invention. FRET occurs between two fluorophores when the excitation of the donor fluorophore is transferred to the acceptor fluorophore. This interaction is dependent on the distance between the donor and acceptor fluorophore and distance-dependent interaction between a donor and acceptor molecule. The donor and acceptor molecules are fluorophores. If the fluorophores have excitation and emission spectra that overlap, then in close proximity (typically around 10-100 angstroms) the excitation of the donor fluorophore is transferred to the acceptor fluorophore. The relative proximity of the first and second labels is determined by measuring a change in the intrinsic fluorescence of the first or second label. Commonly, the emission of the first label is quenched by proximity of the second label.

Many appropriate interactive labels for FRET are known. For example, fluorescent labels, dyes, enzymatic labels, and antibody labels are all appropriate. Examples of preferred interactive fluorescent label pairs include terbium chelate and TRITC (tetrarhodamine isothiocyanate), europium cryptate and allophycocyanin and many others known to one of skill. Similarly, two calorimetric labels can result in combinations that yield a third color, e.g., a blue emission in proximity to a yellow emission produces an observed green emission.

With regard to preferred fluorescent pairs, there are a number of fluorophores which are known to quench each other. Fluorescence quenching is a bimolecular process that reduces the fluorescence quantum yield, typically without changing the fluorescence emission spectrum. Quenching can result from transient excited interactions, (collisional quenching) or, e.g., from the formation of nonfluorescent ground state species. Self-quenching is the quenching of one fluorophore by another; it tends to occur when high concentrations, labeling densities, or proximity of labels occurs. Some excited fluorophores interact to form excimers, which are excited state dimers that exhibit altered emission spectra (e.g., phospholipid analogs with pyrene sn-2 acyl chains); See, Haugland (1996) Handbook of Fluorescent Probes and Research Chemicals, published by Molecular Probes, Inc., Eugene, Org.

The Forster radius (Rₒ) is the distance between fluorescent pairs at which energy transfer is 50% efficient (i.e., at which 50% of excited donors are deactivated by FRET). The magnitude of Rₒ is dependent on the spectral properties of donor and acceptor dyes: Rₒ=[8.8×10²³·K²·n⁻⁴·QY_{D}·J(λ)]^{⅙} Å; where K²=dipole orientation range factor (range 0 to 4, K²=⅔ for randomly oriented donors and acceptors).; QY_{D}=fluorescence quantum yield of the donor in the absence of the acceptor; n=refractive index; and J( )=spectral overlap integral=∫ε_{A}(λ)·F_{D}·(λ4)dλ cm³M⁻¹, where ε_{A}=extinction coefficient of acceptor and FD=fluorescence emission intensity of donor as a fraction of total integrated intensity. Some typical Rₒ are listed for typical donor acceptor pairs in Table 1:

TABLE 1

| Donor | Acceptor | R₀ (Å) |
|---|---|---|
| Fluorescein | Tetramethylrhodamine | 55 |
| IAEDANS | Fluorescein | 46 |
| EDANS | DABCYL | 33 |
| BODIPY FL | BODIPY FL | 57 |
| Fluorescein | QSY-7 dye | 61 |

An extensive compilation of Rₒ values are found in the literature; see, Haugland (1996), supra. In most uses, the donor and acceptor dyes are different, in which case FRET can be detected by the appearance of sensitized fluorescence of the acceptor or by quenching of the donor fluorescence. When the donor and acceptor are the same, FRET is detected by the resulting fluorescence depolarization.

In addition to quenching between fluorophores, individual fluorophores are also quenched by nitroxide-labeled molecules such as fatty acids. Spin labels such as nitroxides are also useful in the liquid phase assays of the invention.

Test compounds and a polypeptide that includes an LXRα ligand binding domain can be labeled with FRET pairs. If the test compound can directly interact with the LXRα ligand binding domain, fluorescence resonance energy transfer can take place and the affinity can be measured. Alternatively, a known LXRα ligand can be labeled with an appropriate FRET label and incubated with an FRET fluorophore labeled polypeptide that includes an LXRα ligand binding domain. Fluorescence resonance energy transfer can take place between the labeled LXRα ligand and the labeled LXRα ligand binding domain. If a test compound were incubated with the two labeled components, the amount of FRET would be lowered if the test compound can inhibit or displace the binding of the labeled LXRα ligand to the LXRα ligand binding domain.

Additional methods for assaying the ability of test compounds to modulate LXRα interactions with its ligands employ peptide sensors. These assays can be adapted from those described in WO 99/27365. Briefly, these assays use a peptide sensor to which is attached a detectable label. The peptides are based on corepressor or coactivator protein motif sequences, either naturally occurring or derived from mutational analysis. The peptide sensors are derived from corepressors or coactivators that interact with LXRα (e.g., RXR, SRC-1 and NCOR, and others that are identified as described herein). Alternatively, the peptides can be obtained through randomizing residues and selecting for binding to the LXRα receptor polypeptide. Panels of predetermined or randomized candidate sensors can be screened for receptor binding. For LXRα peptide sensor assays, an example of a suitable peptide sensor is derived from the receptor-interacting domain of the coactivator SRC-1. This domain has been mapped to a short motif with the amino acid sequence LXXLL, where L is leucine and X is any amino acid. Fragments of SRC-1 or short synthetic peptides containing one LXXLL motif or more bind nuclear receptors in a ligand-dependent manner (Darimont et al., Genes Dev. 12:3343 (1998); McInerney et al., Genes Dev. 12:3357 (1998)). A corepressor-derived peptide sensor can include the motif IXXII, where I is isoleucine and X is any amino acid.

In typical embodiments, the sensor peptides are labeled with a detectable label. The detectable labels can be primary labels (where the label comprises an element that is detected directly or that produces a directly detectable element) or secondary labels (where the detected label binds to a primary label, as is common in immunological labeling). An introduction to labels, labeling procedures and detection of labels is found in Polak and Van Noorden (1997) Introduction to Immunocytochemistry, 2nd ed., Springer Verlag, N.Y. and in Haugland (1996) Handbook of Fluorescent Probes and Research Chemicals, a combined handbook and catalogue published by Molecular Probes, Inc., Eugene, Org. Primary and secondary labels can include undetected elements as well as detected elements. Useful primary and secondary labels in the present invention can include spectral labels such as fluorescent dyes (e.g., fluorescein and derivatives such as fluorescein isothiocyanate (FITC) and Oregon Green™, rhodamine and derivatives (e.g., Texas red, tetrarhodimine isothiocynate (TRITC), etc.), digoxigenin, biotin, phycoerythrin, AMCA, CyDyes™, and the like), radiolabels (e.g., ³H, ¹²⁵I, ³⁵S, ¹⁴C, ³²P, ³³P, etc.), enzymes (e.g., horseradish peroxidase, alkaline phosphatase, etc.), spectral colorimetric labels such as colloidal gold or colored glass or plastic (e.g., polystyrene, polypropylene, latex, etc.) beads. The label may be coupled directly or indirectly to a component of the detection assay (e.g., the detection reagent) according to methods well known in the art. As indicated above, a wide variety of labels may be used, with the choice of label depending on sensitivity required, ease of conjugation with the compound, stability requirements, available instrumentation, and disposal provisions. In a presently preferred embodiment, the detectable label is a fluorescent label, in which case fluorescence polarization detection provides a sensitive and efficient means of detecting whether the peptide sensor is bound to the LXRα receptor polypeptide. See, e.g., Schindler et al., Immunity 2:689-697 (1995)).

The sensor peptide and the LXRα polypeptide are incubated under conditions that are suitable for sensor binding to the receptor polypeptide. In some embodiments, a candidate modulator of LXRα binding to a corepressor, coactivator or other ligand is included in the reaction mixture. If a candidate modulator increases or decreases binding of the sensor peptide to the LXRα polypeptide, the candidate modulator is a potential lead compound for blocking the LXRα-mediated effect on SREBP-1 expression.

2. Cell-based Screening Methods

The present invention also provides cell-based methods for screening to identify compounds that are suitable for use as modulators of SREBP-1 expression, and thus are useful as therapeutic agents. The cell-based screening methods are suitable for initial screening of compounds, or can be used for further screening of compounds that exhibit activity in other assays, such as the polypeptide-based assays described above. These methods provide an assay to determine whether expression of a gene involved in fatty acid and/or triglyceride biosynthesis is affected by administration of the test compound.

In some embodiments, these screening methods of the invention use a cell that contains a polypeptide that has a ligand binding domain (LBD) which is at least substantially identical to that of LXRα. The polypeptide typically will also include a DNA binding domain (DBD). The DBD can be substantially identical to that of LXRα (i.e., a full-length LXRα is used), or substantially identical to that of a receptor other than LXRα for which the response element is known (e.g., GAL4, other nuclear hormone receptors, and the like). Conveniently, the chimeric receptor polypeptide is introduced into the cell by expression of a polynucleotide that encodes the receptor polypeptide. For example, an expression vector that encodes the chimeric receptor can be introduced into the cell that is to be used in the assay. Suitable LXRα polypeptides and chimeric receptors are described below.

The cells generally will also contain a response element to which the particular DNA binding domain can bind. Response elements, including glucocorticoid response elements (GRE) and estrogen response elements (ERE), are described in, for example, Jantzen et al., Cell 49:29 (1987); Martinez et al., EMBO J. 6:3719 (1987) and Burch et al., Mol. Cell. Biol. 8:1123 (1988). Many other response elements are known; a commonly used response element is the GAL4 upstream activating sequence (UAS_{G}) (Keegan et al., Science 14:699-704 (1986)), which is responsive to binding by chimeric receptors that include the GAL4 DNA binding domain.

The response element that is bound by the DNA binding domain used in the chimeric polypeptide is generally used in a reporter gene construct. In such constructs, the response element is operably linked to a promoter that is active in the cell used for the assay. In presently preferred embodiments, the promoter is operably linked to a reporter gene that, when expressed, produces a readily detectable product. The response element/reporter gene construct is conveniently introduced into cells as part of a “reporter plasmid.” However, one can also monitor expression of genes that are naturally under the control of an LXRα response element (e.g., SREBP-1) or genes that are regulated by SREBP-1 (e.g., genes involved in fatty acid synthesis, triglyceride synthesis, and the like.

Suitable promoters include those described herein, and others known to those of skill in the art. In presently preferred embodiments, the promoter is operably linked to a reporter gene that, when expressed, produces a readily detectable product. A variety of reporter gene plasmid systems are known, such as the chloramphenicol acetyltransferase (CAT) and β-galactosidase (e.g., bacterial lacZ gene) reporter systems, the firefly luciferase gene (See, e.g., Cara et al., J. Biol. Chem., 271:5393-5397 (1996)), the green fluorescence protein (see, e.g., Chalfie et al., Science 263:802 (1994)) and many others. Examples of reporter plasmids are also described in U.S. Pat. No. 5,071,773. Selectable markers which facilitate cloning of the vectors of the invention are optionally included. Sambrook and Ausubel, both supra, provide an overview of selectable markers.

In some embodiments of this assay, the reporter plasmid and an expression plasmid that encodes the chimeric receptor are introduced into a suitable host cell. Standard transfection methods can be used to introduce the vectors into the host cells. For mammalian host cells, preferred transfection methods include, for example, calcium phosphate precipitation (Chen and Okayama, BioTechniques 6:632 (1988)), DEAE-dextran, and cationic lipid-mediated transfection (e.g., Lipofectin) (see, e.g., Ausubel, supra.). In some cases, the host cell, prior to introduction of the expression plasmid, should not contain an LXRα receptor. See, e.g., U.S. Pat. No. 5,071,773 for suitable host cells for use in the assays.

The assay methods involve contacting test cells that contain the reporter plasmid and the native or chimeric LXRα polypeptide with a potential modulator compound. In some embodiments, a ligand for LXRα is also provided (e.g., an oxysterol such as 24, 25-epoxycholesterol) in addition to the test compound. An RXR polypeptide and/or other coactivators and corepressors which mediate the effect of LXRα can also be present in the test cells. Cells that contain a reporter gene construct and the chimeric peptide can be grown in the presence and absence of putative modulatory compounds and the levels of reporter gene expression observed in each treatment compared.

The observed effect on reporter gene expression can depend on the particular assay system used. For example, when an LXRα polypeptide that includes the LXRα DBD, AF-2 domain and LBD is used, cells grown in the absence of the LXRα ligand will exhibit a level of reporter gene expression that is lower than the level observed in the absence of the ligand. Conversely, when a GAL4 DBD is used, binding of the ligand to the fusion polypeptide will result in increased expression the reporter gene to which is linked the GAL4 response element. Therefore, reporter gene expression is increased when cells are grown in the presence of a ligand for LXRα.

B. LXRα Polypeptides and Fusion Polypeptides

The assays of the invention typically employ an LXRα polypeptide. The LXRα polypeptide can be a full-length LXRα, or can include one or more domains of LXRα. In some embodiments, one or more LXRα domains (e.g., a DNA binding domain (DBD) or a ligand binding domain (LBD)) are used as a fusion protein with a domain from another polypeptide, such as another receptor. For example, some assay formats use a fusion protein that includes an LXRα LBD fused to a DBD of another receptor.

The LXRα polypeptides and fusion polypeptides used in the assays of the invention can be made by methods known to those of skill in the art. For example, the LXRα proteins or subsequences thereof can be synthesized using recombinant DNA methodology. Generally, this involves creating a DNA sequence that encodes the polypeptide, modified as desired, placing the DNA in an expression cassette under the control of a particular promoter, expressing the protein in a host, isolating the expressed protein and, if required, renaturing the protein.

1. Nucleic Acids that Encode LXRα Polypeptides

LXRα polypeptides, and polynucleotides that encode LXRα polypeptides, are known to those of skill in the art. For example, cDNA sequences of LXRα polypeptides from mouse (GenBank Accession No. AJ132601; Alberti et al., Gene 243:93-103 (2000)), and human (GenBank Accession No. U22662; Willy et al., Genes Dev. 9:1033-1045 (1995)) are found in GenBank. The nucleic acids that encode LXRα can be used to express the LXRα polypeptide, or to construct genes that encode a desired fusion polypeptide.

LXRα-encoding nucleic acids can be isolated by cloning or amplification by in vitro methods, such as the polymerase chain reaction (PCR), the ligase chain reaction (LCR), the transcription-based amplification system (TAS), the self-sustained sequence replication system (SSR). A wide variety of cloning and in vitro amplification methodologies are well-known to persons of skill. Examples of these techniques and instructions sufficient to direct persons of skill through many cloning exercises are found in Berger, Sambrook, and Ausubel (all supra.); Cashion et al., U.S. Pat. No. 5,017,478; and Carr, European Patent No. 0,246,864. Examples of techniques sufficient to direct persons of skill through in vitro amplification methods are found in Berger, Sambrook, and Ausubel, as well as Mullis et al. (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3:81-94; (Kwoh et al., Proc. Nat'l Acad. Sci. USA 86:1173 (1989); Guatelli et al., Proc. Natl. Acad. Sci. USA 87:1874 (1990); Lomell et al., J. Clin. Chem. 35:1826 (1989); Landegren et al., Science 241:1077-1080 (1988); Van Brunt, Biotechnology 8:291-294 (1990); Wu and Wallace, Gene, 4:560 (1989); and Barringer et al., Gene 89:117 (1990).

In one preferred embodiment, LXRα cDNAs can be isolated by routine cloning methods. The cDNA sequences provided in GenBank, for example, can be used to provide probes that specifically hybridize to a LXRα c gene in a genomic DNA sample, to an LXRα mRNA in a total RNA sample, or to a LXRα cDNA in a cDNA library (e.g., in a Southern or Northern blot). Once the target LXRα nucleic acid is identified, it can be isolated according to standard methods known to those of skill in the art (see, e.g., Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel). In another preferred embodiment, the LXRα nucleic acids can be isolated by amplification methods such as polymerase chain reaction (PCR).

A polynucleotide that encodes an LXRα polypeptide or fusion protein can be operably linked to appropriate expression control sequences for a particular host cell in which the polypeptide is to be expressed. For E. coli, appropriate control sequences include a promoter such as the T7, trp, or lambda promoters, a ribosome binding site and preferably a transcription termination signal. For eukaryotic cells, the control sequences typically include a promoter which optionally includes an enhancer derived from immunoglobulin genes, SV40, cytomegalovirus, etc., and a polyadenylation sequence, and may include splice donor and acceptor sequences. In yeast, convenient promoters include GAL1,10 (Johnson and Davies, Mol. Cell. Biol. 4:1440-1448 (1984)) ADH2 (Russell et al., J. Biol. Chem. 258:2674-2682 (1983)), PHO5 (EMBO J., 6:675-680 (1982)), and MFαl (Herskowitz and Oshima (1982) in The Molecular Biology of the Yeast Saccharomyces (eds. Strathem, Jones, and Broach) Cold Spring Harbor Lab., Cold Spring Harbor, N.Y., pp. 181-209).

Expression cassettes are typically introduced into a vector which facilitates entry into a host cell, and maintenance of the expression cassette in the host cell. Vectors that include a polynucleotide that encodes an LXRα polypeptide are provided by the invention. Such vectors often include an expression cassette that can drive expression of the LXRα polypeptide. To easily obtain a vector of the invention, one can clone a polynucleotide that encodes the LXRα polypeptide into a commercially or commonly available vector. A variety of common vectors suitable for this purpose are well known in the art. For cloning in bacteria, common vectors include pBR322 derived vectors such as PBLUESCRIPT™, and λ-phage derived vectors. In yeast, vectors include Yeast Integrating plasmids (e.g., YIp5) and Yeast Replicating plasmids (the YRp series plasmids) and pGPD-2. A multicopy plasmid with selective markers such as Leu-2, URA-3, Trp-1, and His-3 is also commonly used. A number of yeast expression plasmids such as YEp6, YEp13, YEp4 can be used as expression vectors. The above-mentioned plasmids have been fully described in the literature (Botstein et al., Gene 8:17-24 (1979); Broach et al., Gene, 8:121-133 (1979)). For a discussion of yeast expression plasmids, see, e.g., Parents, B., YEAST (1985), and Ausubel, Sambrook, and Berger, all supra). Expression in mammalian cells can be achieved using a variety of commonly available plasmids, including pSV2, pBC12BI, and p91023, as well as lytic virus vectors (e.g., vaccinia virus, adenovirus, and baculovirus), episomal virus vectors (e.g., bovine papillomavirus), and retroviral vectors (e.g., murine retroviruses).

LXRα polypeptides and fusion polypeptides that include at least one LXRα domain can be expressed in a variety of host cells, including E. coli, other bacterial hosts, yeasts, filamentous fungi, and various higher eukaryotic cells such as the COS, CHO and HeLa cells lines and myeloma cell lines. Techniques for gene expression in microorganisms are described in, for example, Smith, Gene Expression in Recombinant Microorganisms (Bioprocess Technology, Vol. 22), Marcel Dekker, 1994. Examples of bacteria that are useful for expression include, but are not limited to, Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus. Filamentous fungi that are useful as expression hosts include, for example, the following genera: Aspergillus, Trichoderma, Neurospora, Penicillium, Cephalosporium, Achlya, Podospora, Mucor, Cochliobolus, and Pyricularia. See, e.g., U.S. Pat. No. 5,679,543 and Stahl and Tudzynski, Eds., Molecular Biology in Filamentous Fungi, John Wiley & Sons (1992). Synthesis of heterologous proteins in yeast is well known and described in the literature. Methods in Yeast Genetics, Sherman, F., et al., Cold Spring Harbor Laboratory (1982) is a well recognized work describing the various methods available to produce the enzymes in yeast.

The nucleic acids that encode the polypeptides of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes, among others. Techniques for transforming fungi are well known in the literature and have been described, for instance, by Beggs et al. (Proc. Nat'l. Acad. Sci. USA 75:1929-1933 (1978)), Yelton et al. (Proc. Natl. Acad. Sci. USA 81:1740-1747 (1984)), and Russell (Nature 301:167-169 (1983)). Procedures for transforming yeast are also well known (see, e.g., Beggs, Nature (London), 275:104-109 (1978); and Hinnen et al., Proc. Natl. Acad. Sci. USA, 75:1929-1933 (1978). Transformation and infection methods for mammalian and other cells are described in Berger, Sambrook, and Ausubel, supra.

Once expressed, the LXRα polypeptides and/or fusion proteins can be purified, either partially or substantially to homogeneity, according to standard procedures of the art, such as, for example, ammonium sulfate precipitation, affinity columns, column chromatography, gel electrophoresis and the like (see, generally, R. Scopes, Protein Purification, Springer-Verlag, N.Y. (1982), Deutscher, Methods in Enzymology Vol. 182: Guide to Protein Purification., Academic Press, Inc. N.Y. (1990)). Once purified, partially or to homogeneity as desired, the polypeptides may then be used (e.g., in screening assays for modulators for gene expression or as immunogens for antibody production).

One of skill in the art would recognize that after chemical synthesis, biological expression, or purification, the LXRα polypeptides and/or fusion proteins may possess a conformation substantially different than the native conformations of the constituent polypeptides. In this case, it may be necessary to denature and reduce the polypeptide and then to cause the polypeptide to re-fold into the preferred conformation. Methods of reducing and denaturing proteins and inducing re-folding are well known to those of skill in the art (See, Debinski et al., J. Biol. Chem., 268:14065-14070 (1993); Kreitman and Pastan, Bioconjug. Chem., 4:581-585 (1993); and Buchner, et al., Anal. Biochem., 205:263-270 (1992)). Debinski et al., for example, describe the denaturation and reduction of inclusion body proteins in guanidine-DTE. The protein is then refolded in a redox buffer containing oxidized glutathione and L-arginine.

One of skill also would recognize that some modifications can be made to the LXRα polypeptides without diminishing their biological activity. Such modifications can be made to facilitate the cloning, expression, or incorporation of the polypeptide into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

2. Chimeric LXRα Polypeptides

The assays of the invention sometimes employ a chimeric LXRα polypeptide that has at least a ligand binding domain (LBD) and a DNA binding domain (DBD). At least one of the ligand binding domain and the DNA binding domain of the chimeric receptors of the invention is substantially identical to the corresponding domain of LXRα. These chimeric receptors are useful for many purposes. For example, one can use the chimeric receptors to identify additional ligands for LXRα and to identify response elements that are responsive to LXRα. The chimeric receptors are also useful in screening assays for identifying compounds that can modulate interactions between LXRα and its ligands and/or response elements.

a. DNA Binding Domains

The chimeric receptors used in the assays of the invention include those having a ligand binding domain that is at least substantially identical to a ligand binding domain of LXRα, as well as those that have a DNA binding domain that is not substantially identical to a DNA binding domain of an LXRα. For example, the DNA binding domain can be about 90% or less identical to that of an LXRα, more preferably about 75% or less, and most preferably about 60% or less identical. Often, the DNA binding domain is derived from a receptor other than LXRα. In a typical embodiment, the DNA binding domain is at least substantially identical to a DNA binding domain from a nuclear hormone receptor or a steroid hormone receptor.

The modular nature of transcription regulators facilitates the construction of chimeric receptors that have domains that are derived from different receptors (Green and Chambon, Nature 324:615-617 (1986)). For example, DNA binding domains derived from steroid, thyroid, and retinoid hormone receptor are suitable for use in the chimeric receptors of the invention. The DNA binding domains of receptors for steroid, thyroid, and retinoid hormones typically include two zinc finger units (Rhodes and Klug, Scientific American, pp. 56-65 (February 1993)). The DNA binding domains of these receptors, are generally cysteine-rich regions of about 65 amino acids that fold into two cysteine-rich “C4” type zinc fingers. The boundaries for many DNA binding domains have been identified and characterized for the steroid hormone superfamily. See, e.g., Giguere et al., Cell 46:645-652 (1986); Hollenberg et al., Cell 49:39-46 (1987); Green and Chambon, Nature 325:74-78 (1987); Miesfield et al., Science 236:423-427 (1987); and Evans, Science 240:889-895 (1988).

Examples of receptors from which one can derive DNA binding domains that are suitable for use in the chimeric receptors of the invention include, for example, androgen receptors, estrogen receptors, glucocorticoid receptors, mineralcorticoid receptors, progesterone receptors, retinoic acid receptors (including α, β (hap), and γ), thyroid hormone receptors (including α and β), the gene product of the avian erythroblastosis virus oncogene v-erbA (which is derived from a cellular thyroid hormone receptor), vitamin D3 receptor, Drosophila ecdysone receptor (EcR), COUP transcription factor (also known as ear3) and its Drosophila homolog 7UP (svp), hepatocyte nuclear factor 4 (HNF-4), Ad4BP, apolipoprotein AI regulatory protein-1 (ARP-1), peroxisome proliferator activated receptor (PPAR), Drosophila protein knirps (kni), Drosophila protein ultraspiracle (usp; chorion factor 1), human estrogen receptor related genes 1 and 2 (err1 and err2), human erbB related gene 2 (ear2), human NAK1/mouse nur/77 (N10)/rat NGFI-B; Drosophila protein embryonic gonad (egon), Drosophila knirps-related protein (knr1), Drosophila protein tailless (tll), Drosophila 20-O-ecdysone regulated protein E75, and Drosophila Dhr3. Some of these and other suitable receptors are described in, for example, Evans, R M, Science 240:889-895 (1988); Gehrig, U., Trends Biochem. Sci. 12:399-402 (1987); Beato, M., Cell 56:335-344 (1989); Laudet et al., EMBO J. 11:1003-1013 (1992).

In some embodiments, the chimeric receptors include a DNA binding domain from a DNA-binding polypeptide other than a nuclear receptor. For example, chimeric receptors that have the DNA binding domain of GAL4, which is a positive regulatory protein of yeast (Giniger et al., Cell 40:767-774 (1985); Sadowski et al., Gene 118:137-141(1992)) linked to a ligand binding domain of an LXRα polypeptide are provided. GAL4 DNA binding domain-containing fusion proteins can be readily expressed by cloning a coding sequence for an LXRα ligand binding domain into a commercially available expression vector that includes a GAL4 DNA binding domain coding sequence under the control of a promoter (e.g., pAS2-1 (CLONTECH Laboratories, Inc.). Another example of a well-characterized DNA binding domain for which expression vectors are commercially available is that of LexA (pLexA, CLONTECH). The chimeric receptors can also include a nuclear localization sequence associated with the DNA binding domain (see, e.g., Silver et al., Proc. Nat'l. Acad. Sci. USA 81:5951-5955 (1984) for a GAL4 nuclear localization sequence).

The chimeric receptors can use an entire receptor molecule as a DNA binding domain, or can use portions of molecules that are capable of binding to nucleic acids, directly or indirectly. To identify such DNA binding domains, one can perform assays such as an electrophoretic mobility shift assay (EMSA) (Scott et al., J. Biol. Chem. 269:19848-19858 (1994)), in which a nucleic acid of interest is allowed to associate with various fragments of a polypeptide to identify those fragments that are capable of binding to the nucleic acid. Association of a portion of the protein with the nucleic acid will result in a retardation of the electrophoretic mobility of the nucleic acid. Another method by which one can identify DNA binding moieties that are suitable for use as DNA binding domains is DNase I footprinting, which is well known to those of skill in the art.

The DNA binding domain can be either a polypeptide or a nucleic acid. Where the DNA binding domain is a nucleic acid, the nucleic acid will be capable of specifically hybridizing to a target nucleic acid site, such as a response element. Hybridization of the nucleic acid to the target site will place the chimeric receptor in a position suitable for activating or repressing expression of a gene that is linked to the target site. An example of an oligonucleotide being chemically linked to a protein by chemical coupling is found in Corey et al., Biochemistry 28:8277-8286 (1989).

These chimeric receptors are useful, for example, in assays to identify modulators of SREBP-1-mediated transcriptional regulation activity as described below.

b. Ligand Binding Domains

The assays of the invention typically use chimeric or non-chimeric LXRα receptors in which the ligand binding domain is at least substantially identical to a ligand binding domain of an LXRα polypeptide.

C. Production of Chimeric LXRα Receptors

To form a chimeric receptor for use in the assay of the invention, the ligand binding domain and the DNA binding domain are linked together. Suitable methods of forming such linkages are known to those of skill in the art. For a review of methods for constructing fusion proteins between receptor ligand binding domains and DNA binding domains, see, e.g., Mattioni et al., Methods in Cell Biology 43(Pt A):335-352 (1994). The linkage can be done using either recombinant or chemical methods. For example, a cysteine residue can be placed at either end of a domain so that the domain can be linked to another domain by, for example, a sulfide linkage. More typically, the ligand binding domains and DNA binding domains are joined by linkers, which are typically polypeptide sequences, such as polyglycine sequences of between about 5 and 200 amino acids, with between about 10-100 amino acids being typical. In some embodiments, proline residues are incorporated into the linker to prevent the formation of significant secondary structural elements by the linker. Preferred linkers are often flexible amino acid subsequences which are synthesized as part of a recombinant fusion protein. In one embodiment, the flexible linker is an amino acid subsequence comprising a proline such as Gly(x)-Pro-Gly(x) where x is a number between about 3 and about 100. A linker can also be a single peptide bond, or one or more amino acid residues. In other embodiments, a chemical linker is used to connect synthetically or recombinantly produced ligand binding domain and DNA binding domain subsequences. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

The chimeric receptors are conveniently produced by recombinant expression in a host cell. Accordingly, the invention provides chimeric nucleic acids that encode a fusion protein that includes a DNA binding domain and a ligand binding domain, at least one of which is at least substantially identical to the corresponding domain of an LXRα of the invention. In some embodiments, the chimeric nucleic acid will also encode a linker region that provides a link between the two domains. Techniques for making such chimeric nucleic acids are known to those of skilled in the art. For example, recombinant methods can be used (see, e.g., Berger and Sambrook, both supra.). Alternatively, the nucleic acid encoding the chimeric receptors can be synthesized chemically.

To obtain expression of a chimeric receptor, a nucleic acid that encodes the chimeric receptor is generally placed under the control of a promoter and other control elements that can drive expression of the chimeric gene in a desired host cell. Accordingly, the invention also provides expression cassettes in which a promoter and/or other control elements are operably linked to a polynucleotide that encodes a chimeric receptor. Suitable promoters, other control sequences, and expression vectors are described above.

C. Assays for LXRα Ligands and Response Elements

Many of the screening assays of the invention utilize ligands for LXRα and/or LXRα response elements. Suitable response elements and ligands are known in the art, and others can be obtained using the following methods.

1. Identification of Ligands for LXRα

The identification of previously unknown LXRα ligands is of particular interest not only for the knowledge obtained regarding the regulation of fatty acid and triglyceride biosynthesis, but also for identifying new compounds that can modulate LXRα-mediated regulation of genes, such as SREBP-1, that are involved in fatty acid and triglyceride biosynthesis.

Candidate ligands include oxysterols and related compounds, and also transcription factors, coactivators, and corepressors with which LXRα might interact. These potential ligands can include other receptor polypeptides (e.g., RXR, coactivators, and the like), which comprise the cellular machinery for regulation of gene expression. For example, nuclear hormone receptors often interact with transcriptional coactivators. Thus, the invention also provides methods of identifying coactivators, corepressors and other molecules that interact with LXRα. These assay methods can involve introducing a coactivator or a corepressor that is a candidate ligand for LXRα into a host cell that contains a chimeric or natural LXRα and a reporter plasmid in which the reporter gene is under the control of an LXR response element. The coactivator can be introduced by means of an expression construct; this expression construct can be present on the same or a different vector than the expression construct for the chimeric receptor.

Ligands for LXRα can be identified using the methods described above for screening to identify compounds that modulate LXRα interactions with the ligands. Instead of including a compound that potentially modulates the interaction, the assays are conducted using a potential LXRα ligand. Both polypeptide-based and cell-based assays can be used.

2. Identification of Response Elements for Transcription Complexes that Include LXRα

The screening methods of the invention involve, in some embodiments, response elements that are responsive to transcription complexes that include LXRα. Methods for obtaining such response elements typically involve contacting a putative response element with a polypeptide that includes an LXRα DNA binding domain (see, e.g., Ausubel et al., supra.). Both cell-based and biochemical methods are available. In presently preferred embodiments of the assays for identification of LXRα response elements, an LXRα receptor, or a chimeric receptor that includes an LXRα DNA binding domain is used. The ligand binding domain of the chimeric receptor is preferably one for which an appropriate ligand is available. Suitable chimeric receptors are described above.

Also provided are methods of identifying response elements to which LXRα does not directly bind. For example, LXRα can form a transcription regulatory complex with one or more other coactivators and/or corepressors. One or more of these other molecules can actually bind to the response element.

Standard gel shift assays can be performed to identify polynucleotides that can bind to a LXRα DNA binding domain. These assays are performed by incubating a polypeptide that includes a LXRα DNA binding domain, either as a purified protein or a complex mixture of proteins) with a labeled DNA fragment that contains the putative LXRα binding site. Reaction products are analyzed on a nondenaturing polyacrylamide gel. To determine the specificity of the binding, one can perform competition experiments using polynucleotides that include a LXRα binding site, or unrelated DNA sequences. Kits for performing gel shift assay include, for example, Gel Shift Assay Systems (Promega, Madison Wis., Part No. TB110).

Another in vitro assay for identifying LXRα response elements is the binding site selection method (see, e.g., U.S. Pat. No. 5,582,981). In this method, a library of oligonucleotides having a randomized nucleotide sequence of about 18 nucleotides flanked by two known nucleotide sequences of sufficient length to allow hybridization to PCR primers that are complementary to these regions. The oligonucleotides are end-labeled (e.g., with γ-³²P) and contacted with a LXRα DNA binding domain polypeptide. A low stringency gel shift experiment is performed. PCR amplification is then carried out on those oligonucleotides to which the LXRα DNA binding domain bound, as evidenced by retardation in the gel shift electrophoresis. Preferably, the selection and amplification process is repeated at least twice more using the amplified fragments.

In vivo assays for LXRα response elements are also provided. The in vivo assays are particularly suitable for confirming results obtained in an in vitro assay. Cells are provided which contain a reporter construct that contains the putative response element in a position relative to a promoter at which binding of an LXRα polypeptide can increase or decrease expression of an operably linked gene. The putative response element can be, for example, a member of a library of polynucleotide fragments. The chimeric receptor and the reporter constructs are introduced into a host cell. Suitable host cells are described in, for example, U.S. Pat. No. 5,071,773. The host cells that contain the reporter plasmid construct and the chimeric receptor are grown in the presence of the ligand for the ligand binding domain used in the chimeric receptor. Those cells in which expression of the reporter gene in the presence of the ligand is greater or less than the expression in the absence of the ligand contain a reporter construct that includes a putative response element for an LXRα. The response elements can be isolated from these cells by, for example, plasmid recovery, PCR amplification, or other methods known to those of skill in the art. Upon isolation, the response elements can be characterized (e.g., by sequencing) and used to identify additional genes for which expression is influenced by LXRα.

D. Compositions Kits and Integrated Systems

The invention provides compositions, kits and integrated systems for practicing the assays described herein. For example, the invention provides an assay system that includes an LXRα polypeptide and a ligand for LXRα. Also provided are assay systems for cell-based screening to identify SREBP-1-modulating compounds. Such systems typically include an expression vector for a full-length or chimeric LXRα polypeptide, a vector that contains an appropriate reporter gene under the control of a transcription complex that includes LXRα, and a suitable host cell is provided by the present invention. Ligands that bind to the ligand binding domain of LXRα can also be included in the assay compositions, as can modulators of LXRα activity.

The invention also provides kits for practicing the LXRα assay methods noted above. The kits can include any of the compositions noted above, and optionally further include additional components such as written instructions to practice a high-throughput method of assaying for LXRα activity, or screening for an inhibitor or activator of LXRα activity, one or more containers or compartments (e.g., to hold reagents, nucleic acids, or the like), and a control LXRα activity modulator.

The invention also provides integrated systems for high-throughput screening of potential SREBP-1 modulators for an effect on binding of LXRα to ligands for LXRα. In other some systems, the modulation of expression of genes that are under the control of SREBP-1, such as the genes involved in fatty acid and triglyceride biosynthesis, is tested. The systems typically include a robotic armature which transfers fluid from a source to a destination, a controller which controls the robotic armature, a label detector, a data storage unit which records label detection, and an assay component such as a microtiter dish comprising a well having a reaction mixture or a substrate.

E. Pharmaceutical Compositions and Methods for Treating Hypertriglyceridemia, Lipodystrophy, and Other Disorders

SREBP-1-mediated disorders and conditions, such as hypertriglyceridemia, lipodystrophy, hyperglyceremia, diabetes, and the like, can be treated with therapeutic agent(s) identified using the methods described herein. The therapeutic agent is typically prepared as a pharmaceutical composition and is administered to a subject suffering from an SREBP-1-mediated disorder or condition.

1. Pharmaceutical Compositions

Accordingly, the present invention provides pharmaceutical compositions that include a pharmaceutically acceptable carrier or excipient and a therapeutic agent. Pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The packaging of the pharmaceutical compositions can also include written instructions for using the compositions for the treatment of conditions such as hypertriglyceridemia, lipodystrophy, and other conditions that are characterized by abnormalities in fatty acid and triglyceride biosynthesis.

The quantity of active component in a unit dose preparation may be varied or adjusted from 0.1 mg to 1000 mg, preferably 1.0 mg to 100 mg according to the particular application and the potency of the active component. The composition can, if desired, also contain other compatible therapeutic agents.

2. Treatment Regime Using Therapeutic Agents

The present invention also provides methods of modulating SREBP-1 activity in a cell. In this aspect, a cell is contacted with an SREBP-1-modulating amount of a compound or composition described above. An SREBP-1-modulating amount can be readily determined using the assays described briefly above, or alternatively, using the assays in the Examples below. The therapeutic agents are especially useful in the treatment of hypertriglyceridemia, lipodystrophy, and other conditions associated with abnormal expression of SREBP-1.

In some embodiments, the present invention provides methods of treating conditions modulated by SREBP-1 in a host animal, by administering to the host an effective amount of a compound or composition provided above. In therapeutic applications, the compounds of the present invention can be prepared and administered in a wide variety of oral and parenteral dosage forms. Thus, the compounds of the present invention can be administered by injection, that is, for example, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally.

The compounds utilized in the pharmaceutical methods of the invention are administered at the initial dosage of about 0.001 mg/kg to about 100 mg/kg daily. A daily dose range of about 0.1 mg/kg to about 10 mg/kg is preferred. The dosages, however, may be varied depending upon the requirements of the patient, the severity of the condition being treated, and the compound being employed. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

Preferably, the therapeutic agents will, upon administration to the subject, cause total triglyceride levels to decrease about 10%, more preferably a decrease of about 20%, and most preferably a decrease of about 25-45%. Triglyceride levels are generally classified as distinct hypertriglyceridemia (triglyceride level>500 mg/dL) and borderline hypertriglyceridemia (triglyceride level 250 to 500 mg/dL). In preferred embodiments, the methods of the invention result in a reduction of triglyceride level to or below 500 mg/dL, and more preferably to or below 250 mg/dL. Still more preferably, triglyceride levels are reduced to or below 200 mg/dL, and even more preferably to or below 100 mg/dL. Once triglyceride levels are reduced to an appropriate level the compositions can then be administered to maintain triglyceride levels at the desired level.

Typically, the host or subject in each of these methods is human, although other animals can also benefit from the foregoing treatments.

### EXAMPLES

The following example is offered to illustrate, but not to limit the present invention.

A. SREBP-1C and Fatty Acid Synthase Expression is Stimulated by LXRα

This Example demonstrates that a ligand of LXRα is a potent stimulator of SREBP-1c and fatty acid synthase expression, but does not affect expression of SREBP-2 or glyceraldehyde-3-dehydrogenase (GAPDH). HepG2 cells were treated with 24,25-epoxycholesterol in the presence or absence of 50 μg/ml cyclohexamide in DMEM F12 media supplemented with 10% lipid-free serum for 18 hours.

Total RNA was then prepared using the TRIzol® method (Chomczynski and Saachi (1987) Anal. Biochem. 162:156). Electrophoresis and Northern blot analysis were performed using the NorthernMax™ system (Ambion). Ribonucleotide probes were used to identify the RNA transcripts.

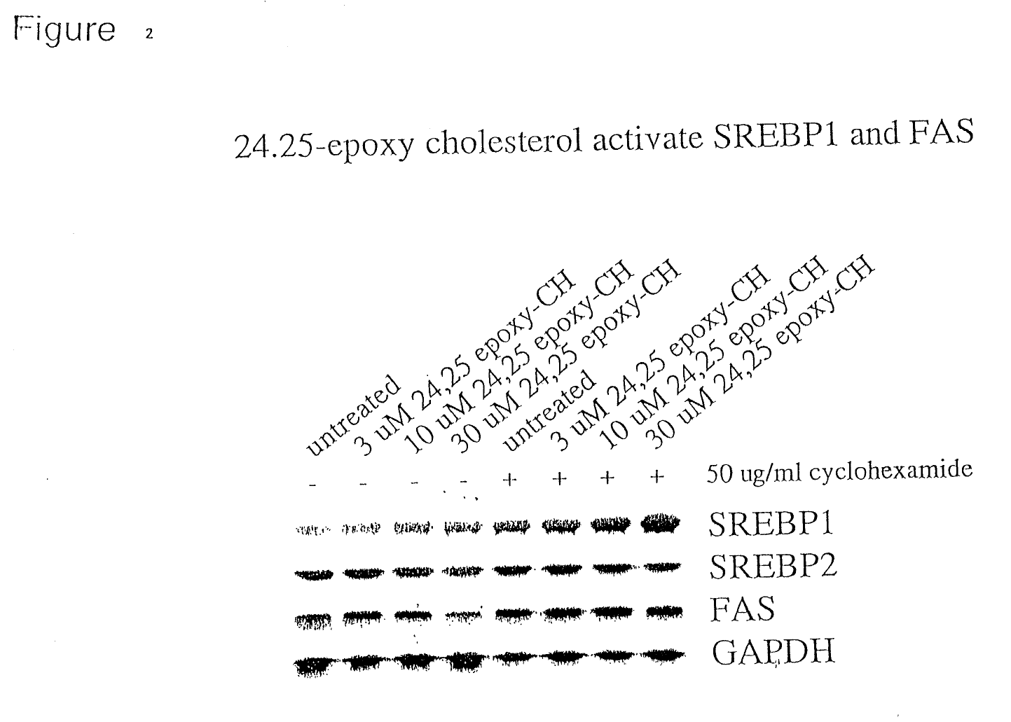

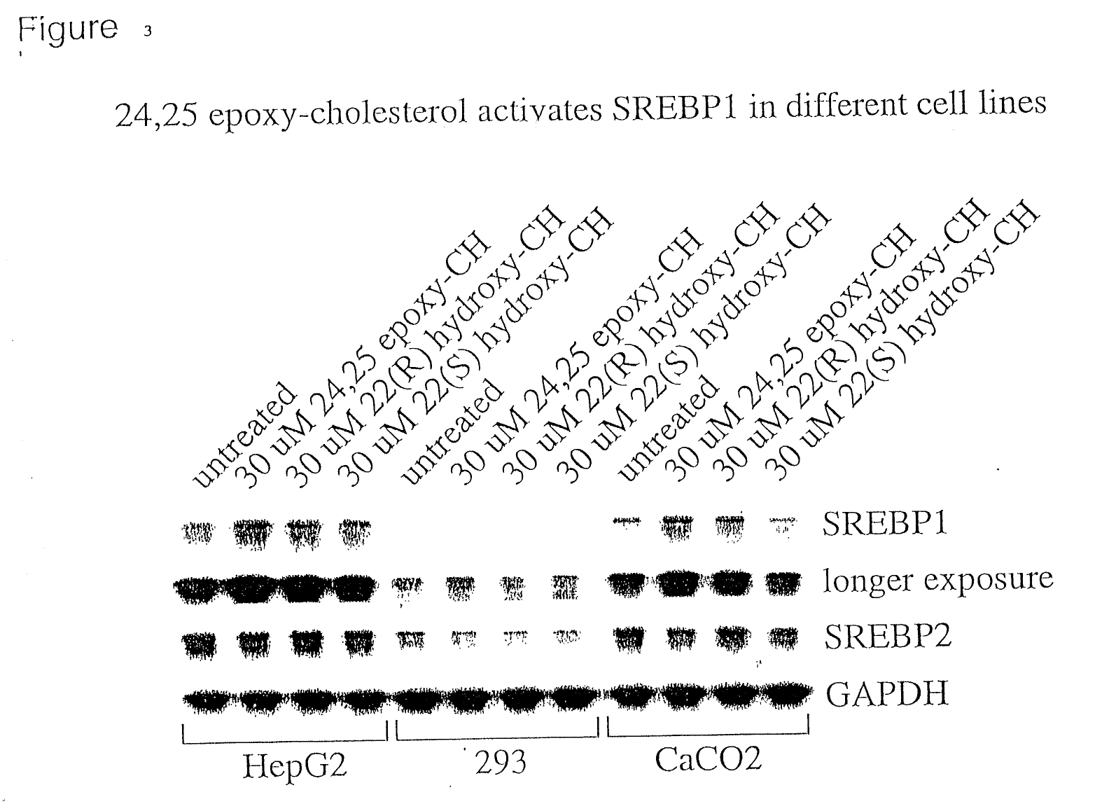

As shown in FIG. 2, expression of SREBP-1 and fatty acid synthase were stimulated by 24,25-epoxycholesterol, both in the presence and absence of cyclohexamide. Expression of SREBP-2 and of the negative control GAPDH was not affected.

B. The LXRα Ligand 24,25-epoxycholesterol Activates SREBP-1 Expression in Different Cell Lines

To ascertain whether or not the 24,25-epoxycholesterol-mediated activation of SREBP-1 expression is cell-specific, three cell lines were tested. HepG2, 293, and CaCO₂ cells were treated with either DMSO, 30 μM of 24,25-epoxycholesterol, 30 μM of 22(R)-hydroxycholesterol, or 30 μM 22(S)-hydroxycholesterol in the presence of 50 μg/ml cyclohexamide in DMEM F12 media supplemented with 10% lipid-free serum for 18 hours.

Total RNA was then prepared using the Trizol method. Electrophoresis and Northern blot analysis were performed using the NorthernMax™ system (Ambion). Ribonucleotide probes were used to identify RNA transcripts.

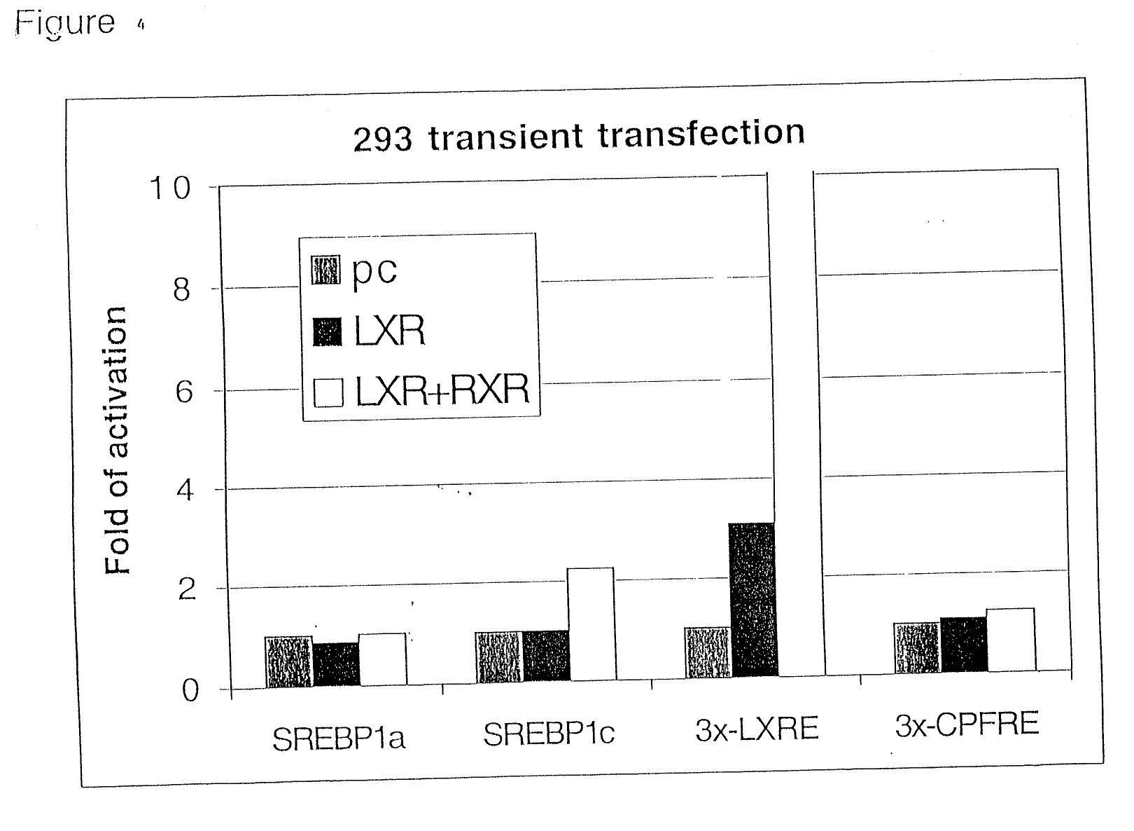

As shown in FIG. 3, SREBP-1 expression was activated by 24,25-epoxycholesterol in all three cell lines. In contrast, expression of SREBP-2 and GAPDH was not significantly enhanced by 24,25-epoxycholesterol.

C. LXR and RXR Activate SREBP-1C Expression

This experiment demonstrates the effect of LXRα and RXR on expression of SREBP-1a and SREBP-1c. 293 cells were transfected with the pGL3 luciferase reporter constructs containing promoters of either SREBP-1a, SREBP-1c, 3X-LXRE-TK, or 3X-CPFRE-TK, along with either pcDNA3 (empty expression vector), pcDNA3-LXR, or pcDNA3-LXR and pcDNA3-RXR. Cells were incubated for 24 hours before being analyzed for luciferase and β-galactosidase activities. Relative luciferase unit (RLU) indicates the luciferase activities normalized to β-galactosidase activity.

As shown in FIG. 4, the combination of LXR and RXR activate expression of SREBP-1c, but not SREBP-1a.

D. Role of LXRS in Control of Lipogenesis

In this example, the identification and bio-chemical features of a nonsteroidal LXR agonist, T0314407, and its analog T0901317, is described. The in vivo role of LXR in lipid metabolism was extended by induction of LXR-regulated pathways in mice and hamsters. It has been shown that LXR agonist treatment induces the expression of genes associated with fatty acid biosynthesis, and it raises plasma triglyceride levels in these animal models. Administration of T0901317 to mice lacking both the LXR α and β genes (LXRα/β−/−) corroborated both the requirement of LXRs in the activation of lipogenesis and their being key components of the triglyceride response. The data presented are consistent with the hypertriglyceridemic effect being associated with LXR agonist-dependent induction of the SREBP-1 lipogenic program.

1. Materials and Methods

### Peptide Sensor Assay

The LXRα ligand-binding domain was fused to the C-terminus of glutathione S-transferase (GST) and the resultant GST-LXR protein was expressed in Escherichia coli and purified on glutathione beads. Rhodamine-labeled peptide (10 nM; with amino acid sequence ILRKLLQE) was incubated on a shaker for 1 h with 400 aM GST-LXR and the indicated compounds in 100 μL of buffer (10 mM Hepes, 150 mM NaCl, 2 mM MgC12, 5 mM DTT at pH 7.9) in a 96-well plate. Fluorescence polarization (mP) was measured on an LJL analyst (LJL Biosystems).

Reporter Gene Assay

HEK293 cells were cotransfected with a luciferase reporter gene and the various Gal4-nuclear receptor chimeric constructs shown and a β-galactosidase (β-gal) expression vector for normalization. Transfected cells were treated with the indicated compounds for 20 h before being harvested. Transfection data and luciferase results are normalized to β-gal and expressed as the fold-induction relative to DMSO controls.

Plasma Lipid Analysis

C57BL/6 mice (Charles River Laboratories) were between 6 and 10 wk old and weighed ˜20-30 g at the initiation of treatment. Golden Syrian Hamsters (Harlan Sprague-Dawley) were between 12 and 16 wk old (80-150 g). Animals were fed regular chow diets containing ˜4% fat and 0.02% cholesterol (PMI Pi-colab 5053 Chow). LXRα and β−/− mice were reared at the University of Texas Southwestern (UTSW) Medical Center at Dallas in accordance with the Institutional Animal Care and Research Advisory Committee at the UTSW Medical Center. Mice at UTSW were maintained on a 12 h light/12 h dark cycle and fed ad libitum a cereal-based mouse chow diet (No. 7001, Harlan Teklad).

Plasma was prepared from euthanized mice using standard centrifugation techniques and analyzed for plasma total cholesterol, HDL cholesterol, and triglyceride concentrations using a Hitachi 704 Clinical Analyzer. FPLC of plasma lipoproteins was accomplished using 200 μL aliquots of pooled plasma (from three to five animals) and fractionated on Superose 6 columns (Pharmacia). Cholesterol, triglyceride, and phospholipid concentrations in the fractions were determined enzymatically with reagents from Boehringer, Sigma, and Wako, respectively. Hepatic lipids were extracted and analyzed for triglyceride essentially as described (Bucolo, G. and David, H., Clin. Chem. 19:476-482 (1973); Yokode et al., Science 250:1273-1275 (1990)).

### Gene Expression

Total RNA extraction and purification for Northern blot analysis was accomplished using the TRI reagent (Molecular Research Center). For GEM analysis, mice were treated for 7 d with and without T0901317 (5 mg/kg) p.o. Total RNA from liver, small intestine (jejunum), and kidney of control (n=3) and agonist-treated animals (n=3) was isolated, pooled, subjected to two rounds of oligo d(T) cellulose chromatography, converted into cDNA, and hybridized to Incyte's mouse GEM1 microarrays containing ˜8000 IMAGE cDNA clones (http://www.incyte.com/reagents/catalog.jsp?page=gem/products/mousegem1).

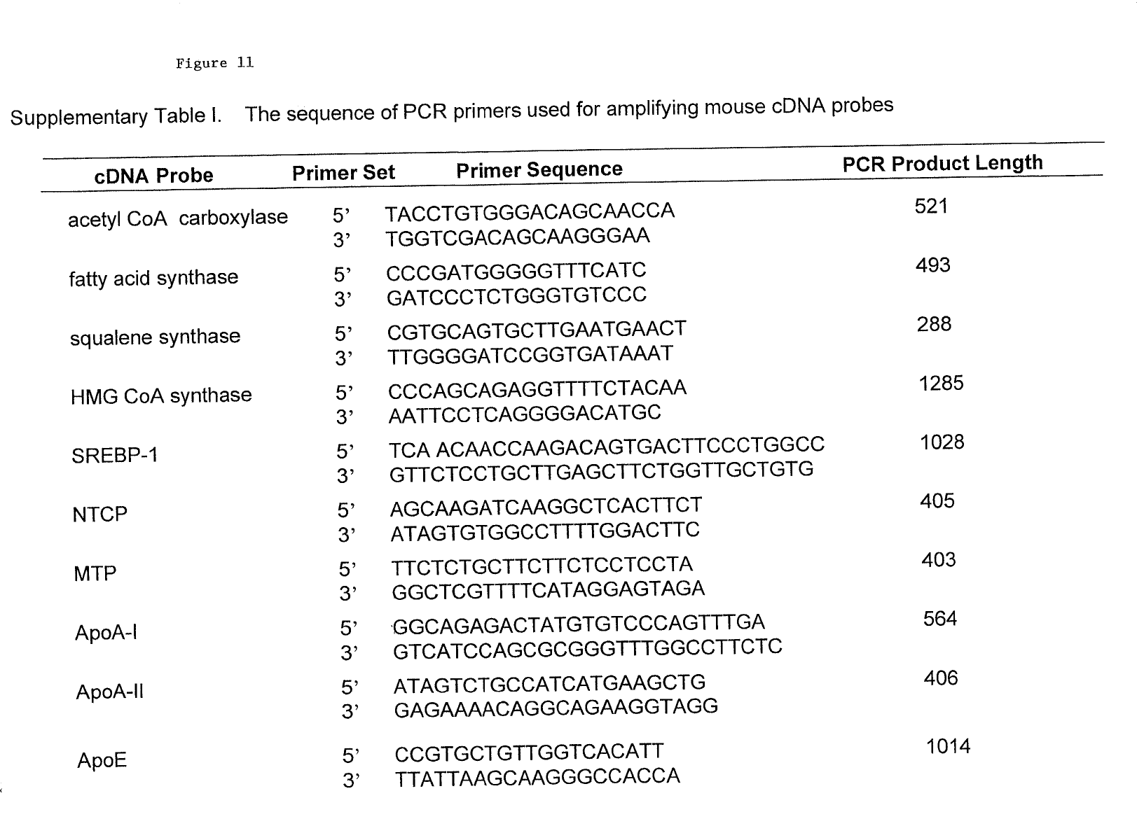

Mouse gene expression was assessed by Northern blot analysis using random primed, ³²P-labeled, cDNA probes that were generated using the primers described in FIG. 11. Hybridization signals were quantified using a phosphoimager (Molecular Dynamics) and standardized against GAPDH controls. Mouse cDNA probes for the numerous genes analyzed were prepared using primers from mouse liver or intestinal cDNA as template. The SREBP-2 cDNA was a gift from J. D. Horton (University of Texas Southwestern Medical Center, Dallas); mouse and hamster CYP7A1 cDNAs were generously supplied by D. W. Russell (University of Texas Southwestern Medical Center, Dallas). SCD-1 was a gift from J. Ntambi (University of Wisconsin, Madison), and apoD was kindly provided by E. Rassart (University of Quebec, Montreal).

For the Northern blot analysis from cycloheximide-treated HepG2 cells, tissue cultures were incubated in cell culture media with 10% lipid-deficient serum with or without 50 μg/mL cycloheximide. T0314407 was added after 30 min of cycloheximide treatment, cells were harvested 18 h later, and total RNA was extracted as described.

2. Results

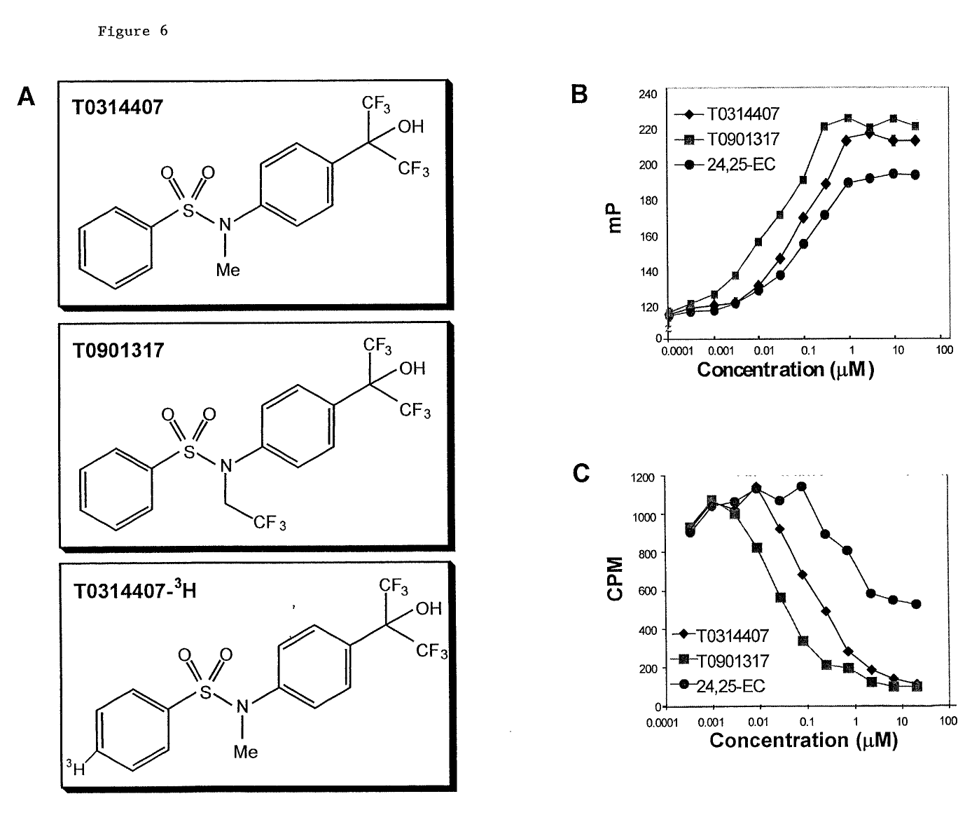

On agonist binding, nuclear receptors undergo a conformational change that increases their affinity for coactivators. Recruitment of coactivator to agonist-bound nuclear receptor is a critical step in the formation of an active transcription complex on DNA. Studies have demonstrated that coactivator fragments containing the motif LXXLL, where L is leucine and X is any amino acid, bind to nuclear receptors in an agonist-dependent manner (Heery et al., Nature 387:733-736 (1997)). In this study, a short synthetic rhodamine-labeled peptide containing an LXXLL motif was synthesized and used it to develop a fluorescence polarization assay for agonist binding to LXRα. In this homogeneous biochemical assay, the greater the extent of rhodamine-peptide binding to LXRα, the greater the extent of fluorescence polarization observed. Thus, addition of an endogenous LXR ligand, 24(S),25-epoxycholesterol (24,25-EC), to a mixture of LXRα protein and rhodamine-peptide led to a dose-dependent increase in fluorescence polarization (FIG. 6B). The EC₅₀ determined for 24,25-EC of 300 nM is in agreement with the Kᵢ determined by a direct ligand-binding assay (Jankowski et at 1999). Screening of >300,000 compounds using this peptide sensor assay led to the identification of T03 14407 (N-methyl-N-[4-(2,2,2-trifluoro-1-hydroxy-1-trifluoromethyl-ethyl)-phenyl]-benzenesulfonamide) as an initial lead (FIG. 6A). A derivative of T0314407 with improved pharmacological properties, T0901317 (N-(2,2,2-trifluoro-ethyl)-N-[4-(2,2,2-trifluoro-1-hydroxy-1 -trifluoromethyl-ethyl)-phenyl]-benzenesulfonamide), was developed through structure-activity relationship studies (FIG. 6A). Both T0314407 and T0901317 showed similar efficacy to 24,25-EC but were significantly more potent and bound to LXRα with EC₅₀ values of 100 and 20 nM, respectively (FIG. 6B).

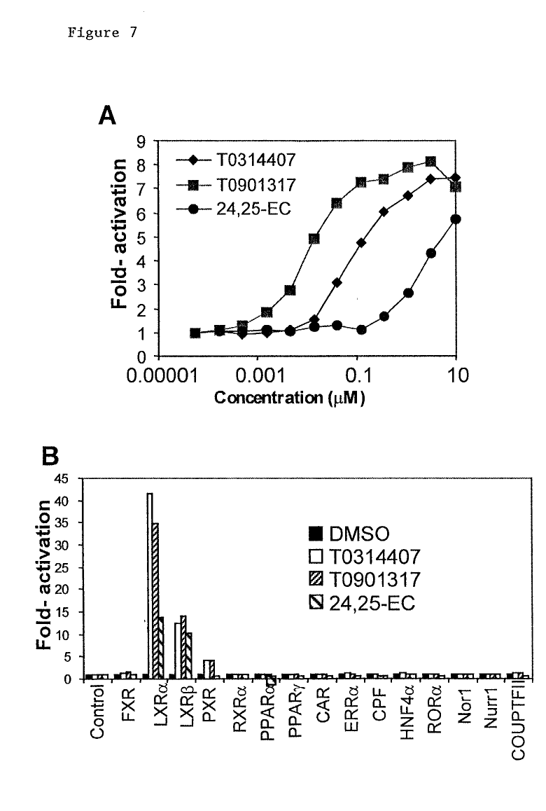

To provide further evidence that these compounds bind directly to LXRα, a scintillation proximity assay (SPA) was developed with radiolabeled T0314407 (FIG. 6C). Unlabeled T0314407 and T0901317 effectively competed for the binding of radiolabeled T0314407 to LXRα in the SPA with IC₅₀ values of 100 and 20 nM, respectively. 24,25-EC also competed with the binding. These results demonstrated direct binding of the synthetic ligands to LXRα and suggested that both the synthetic and endogenous ligands bind at the same site on LXRα. These compounds were then tested for their ability to activate the receptor in a reporter gene assay. HEK293 cells were transiently transfected with an expression plasmid for the human LXRα and a reporter plasmid containing two copies of an LXP response element (Willy et al., Genes & Dev. 9:1033-1045 (1995)) arid then treated with increasing concentrations of T0314407, T0901317, or 24,25-EC (FIG. 7A). Both synthetic ligands induced transcriptional activity of LXRα nearly eightfold with EC₅₀ values of ˜100 and 20 nM, respectively. Consistent with the results obtained in the direct binding assays, T0314407 and T0901317 appeared significantly more active than the endogenous ligand, 24,25-EC, which displayed an EC₅₀ value of −3 μM.

To determine the specificity of TOS14407 and T0901317, a similar transient transfection assay was employed that used chimeric receptors between the yeast transcription factor Gal4 and the nuclear receptor ligand-binding domain (FIG. 7B). Transcriptional activation by T0314407 and T0901317 was selective to LXRs, as these compounds failed to enhance the activity of most other nuclear receptors examined. While T0314407 and T0901317 showed the highest activity against LXRα, transactivation was also noted for chimeric Gal4-LXRβ and, to a lesser extent, Gal4-PXR (pregnane X receptor). PXR, a nuclear receptor involved in the CYP3A-mediated metabolism of a diverse collection of xenobiotics, is known to be activated by a large number of synthetic compounds (Jones et al., Mol. Endocrinol. 14:27-39 (2000)).

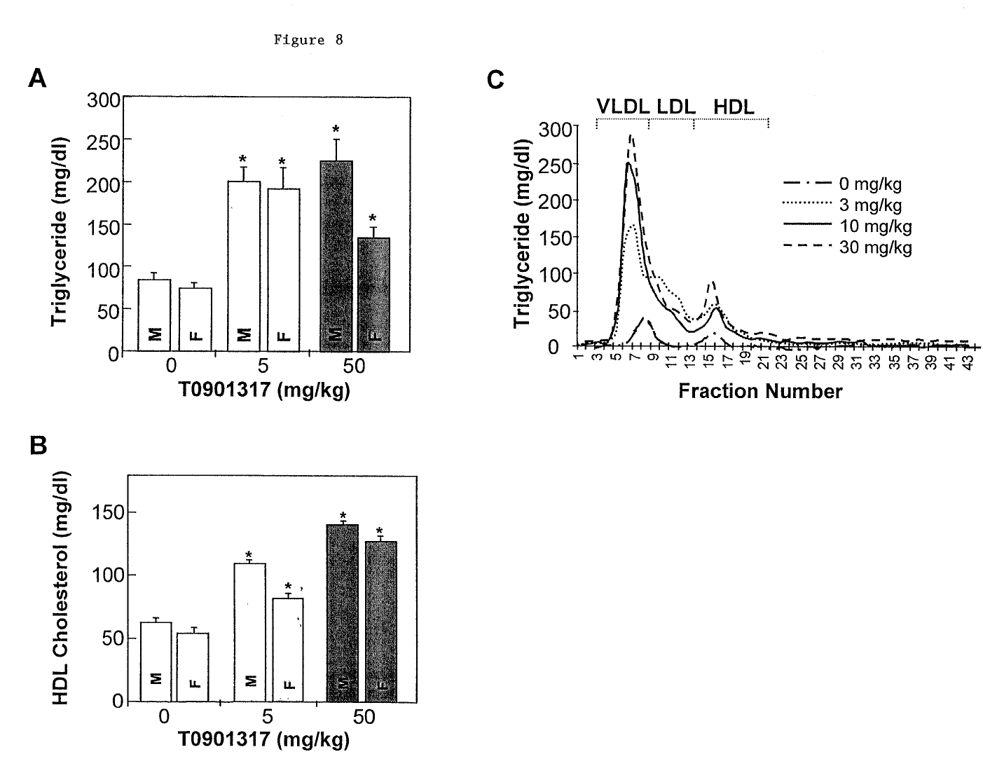

In vitro characterization established T09018 17 as a highly potent and selective nonsteroidal LXRα ligand. To determine the compound's effects on plasma lipid homeostasis, T0901317 was evaluated in small-animal models. Oral treatment of C57BL/6 mice with T09013 17 resulted in significant elevations of plasma triglycerides (FIG. 8A). Increases in plasma very low density lipoproteins (VLDL) triglycerides occurred with comparable increases in plasma total cholesterol (mainly high-density lipoproteins [HDL] cholesterol; FIG. 8B) and HDL-phospholipid. In contrast to humans, which use low-density lipoproteins (LDL) for the transport of most plasma cholesterol, nice carry a preponderance of their cholesterol on HDL. Accordingly, T0901317 was also tested in hamsters, which have lipoprotein cholesterol distributions that more closely resemble human profiles. Similar increases in plasma triglycerides were quantified in the hamster (FIG. 8C). Separation of plasma lipoproteins by fast protein liquid chromatography (FPLC) in combination with analysis of the lipid content across the FPLC fractions further established the increase in triglycerides and demonstrated a principal expansion of the VLDL fraction in mice and hamsters (FIG. 8C).

To understand the mechanism whereby LXR agonist increased plasma triglycerides, changes in global gene expression in response to LXR agonist treatment were assessed by gene expression micrarrays (GEM) and Northern blot analysis. The GEM experiments were carried out using RNA derived from control and LXR agonist-treated HepG2 cells and mice. T09013 17 significantly modulated the expression of a small number of genes in both cell culture and the mouse. Among the genes that showed up-regulation, significant induction was noted for fatty acid synthase and SREBP-l (greater than a twofold increases). In addition, a prominent subset of >20 fatty acid metabolism-associated genes was increased by LXR agonist. This subset included fatty acid synthase, carnitine palmitoyltransferase 1, acyl-CoA-binding protein, acyl glycerol-3 phosphate acyltransferase, fatty acid amide hydrolase, acyl carrier protein, fatty acid binding and transport proteins, and colipase, a small protein cofactor required by lipase for the efficient hydrolysis of dietary lipid. Up-regulation of liver and intestinal phosphoethanolamine cytidylyltransferase, a key regulatory enzyme in the CDP-ethanolamine pathway for phosphatidylethanolamine synthesis, was also noted.

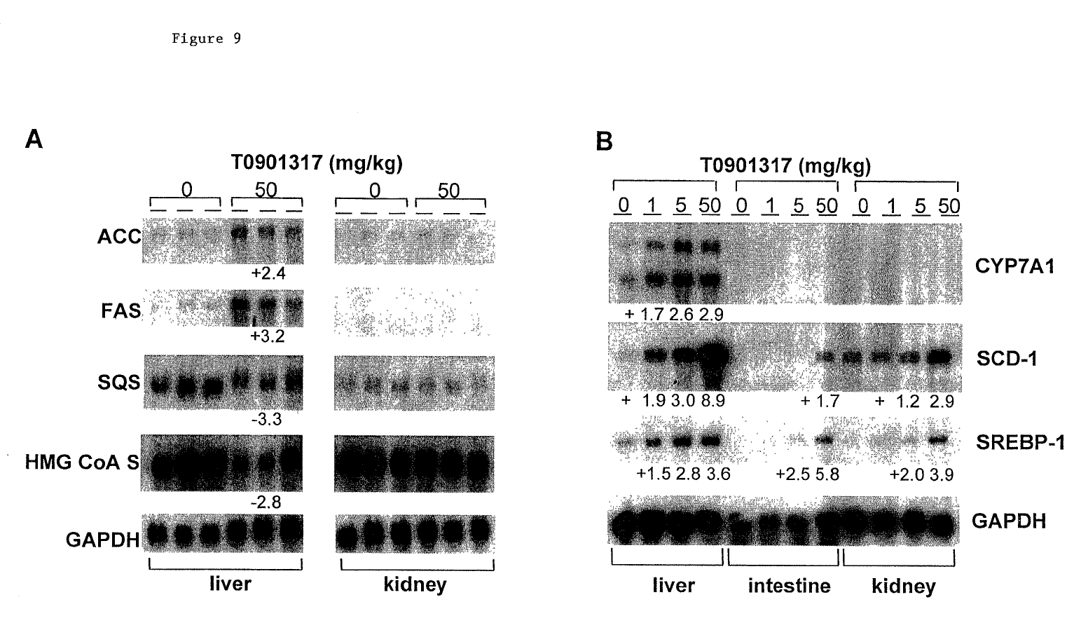

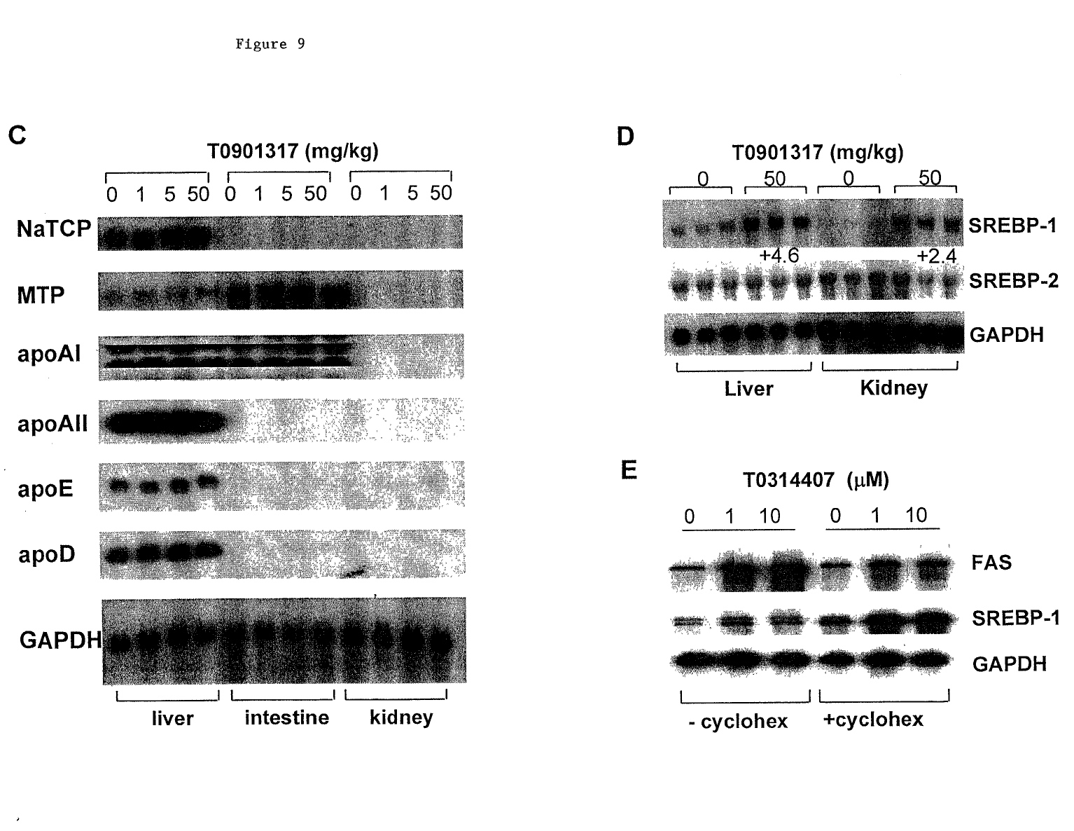

Additional characterization of gene expression was performed by Northern blot analysis using RNA isolated from mice and hamsters (FIG. 9). In the mouse, increases in hepatic fatty acid biosynthetic gene activity were noted after treatment with T0901317, including ACC (twofold), FAS (threefold) and SCD-1 (up to ninefold; FIG. 9A,B). The induction of fatty acid biosynthetic gene activity by T09013 17 is consistent (though diametrically opposed) with the reduction in expression of a similar subset of genes in LXRα-null mice (Peet et al., Cell 93:693-704 (1998)). Steady-state mRNA levels of CYP7A1, SCD-1, and SREBP-1 increased in a dose-dependent manner (FIG. 9B). In contrast, the transcript levels of genes associated with cholesterol biosynthesis such as squalene synthase (SQS) and 3-hydroxy-3-methylglutaryl coenzyme A synthase (HMG CoA S) were consistently reduced after treatment with T09013 17 (FIG. 9A). A subset of genes associated with lipid metabolism remained unaffected by LXR agonist treatment, including apolipoprotein (apo)Al, apoAII, apoD, apoE, microsomal triglyceride transport protein (MTP), and sodium taurocholate cotransporter (NaTCP; FIG. 9C). Other transcripts that also did not appear to vary in level included the scavenger receptor-BI (SR-BI), apoCIII, and acyl CoArdiacylglyerol transferase (DCAT, data not shown).

As described above, increased fatty acid biosynthetic gene activity was accompanied by increases in hepatic SREBP-1 steady-stare mRNA levels (approximately two-to fivefold). In comparison to the relatively high induced levels of SREBP-1, the amount of SRBBP-2 mRNA remained unchanged alter LXR agonist treatment (PIg. 4D). To determine if LXR directly modulates SREBP-1 gene expression, or if target gene activation requires de novo protein synthesis, a series of Northern blots were carried out using RNA prepared from T0314407-dosed HepG2 cells treated both with and without cyeloheximide, an inhibitor of protein synthesis (FIG. 9E). It was found that, while cycloheximide arrested the agonist-mediated increase in levels of FAS mRNA, induction of SREBP-1 gene expression persisted. This outcome suggests that the effect of LXR agonist on SREBP-1 is a primary and direct consequence of LXR agonist-activated transcription. In contrast, activation of FAS and other downstream SREBP-1 targets is likely to be secondary to the stimulation by SREBP-1.

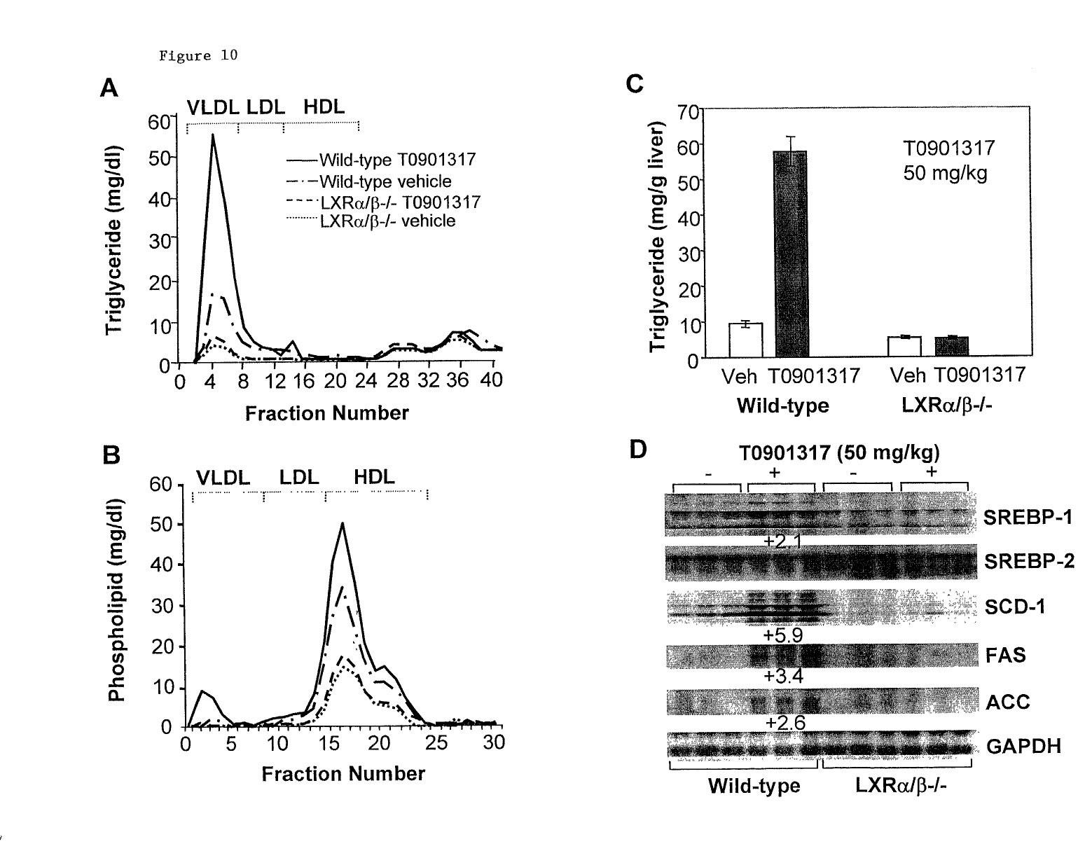

To further validate the T0901317-mediated lipid changes and gene regulation, LXRα/β−/− mice were administered with the LXR agonist, and plasma and hepatic triglyceride levels were quantified. FPLC and lipid measurements showed that T090J317-treated mice (wild type) had an approximate threefold increase in plasma VLDL-triglyceride (FIG. 10A), in comparable agreement with the increase in total plasma triglycerides measured in control and T0901317-treated (wild-type) mice (82.7±9.5 mg/dL and 225.7±7 mg/dL, respectively). The baseline VLDL-triglyceride level in LXRα/β−/− mice was significantly reduced (approximately fourfold) compared to control mice. LXR agonist treatment of the LXRα/β−/− mice only led to a relatively minute increase in plasma VLDL-triglyceride. This result was in agreement with total plasma triglyceride measurements, which also showed only scant increases in T0901317-treated LXRα/β−/− mice compared with vehicle-treated LXRα/β−/− animals. HDL-phospholipid profiles were also LXR-dependent, and similar to those described for VLDL triglyceride (FIG. 10B). Analysis of hepatic triglyceride levels showed a significant fivefold elevation of hepatic triglyceride in wild-type mice compared to vehicle-treated controls (FIG. 10C). Hepatic triglyceride levels were significantly lower in LXRα/β−/− mice, and no significant elevation resulted from treatment with T0901317. The lack of regulation of the SREBP-1, SCD-1, FAS, or ACC gene in response to LXR agonist in the deficient mice was also consistent with the plasma and hepatic lipid characterization (Figure SD). Thus, compared to the robust response of wild-type mice, administration of LXR agonist T0901317 to LXR-null mice had little or no effect on either the lipid profile or the expression of genes involved in lipid metabolism. These results clearly demonstrated that the lipid changes mediated by the synthetic LXR agonist were indeed LXR-dependent.

The current understanding of the control of cholesterol and fatty acid biosynthesis has largely been ascribed to posttranscriptional events associated with the regulation of SREBP processing (Brown, M. S. and Goldstein, J. L., Cell 89:331-340 (1997); Horton et al., Curr. Opin. Lipidol. 10:143-150 (1999)). The current studies suggest an additional primary level of transcriptional regulation that is also available to the cell, one controlled by LXR-mediated induction of SREBP-1 and its associated downstream lipogenic program. The present results support a mechanism whereby LXR directly activates SPEBP-1 gene transcription, presumably through an LXR response element (LXRE), and where subsequent activation of lipogenic genes such as FAS, ACC, and SCD-1 occurs secondary to SREBP-1 gene activation. Indeed, control of SREBP-1 gene expression through an LXRE has recently been established and RNase protection assays revealed induction of the SREBP-1c isoform by LXR agonist (Repa et al., Genes & Dev. 14:2819-2830 (2000a)). Functional LXREs have also been shown to exist in a number of genes associated with cholesterol metabolism, including the rate-limiting enzyme in the classical bile acid biosynthetic pathway CYP7A1 (Lehmann et al., J. Biol. Chem. 272:3137-3140 (1997)), cholesterol ester transferase protein (CETP; Luo, Y. and Tall, A. R., J. Clin. Investig. 105:513-520 (2000)) and the ATP binding cassette (ABC) transporter, ABCA1 (Costet et al., J. Biol. Chem. 275:28240-28245 (2000)) gene promoters. The oxysterol-mediated induction of human white (murine ABC8) gene expression also appears to be mediated by LXRs (Venkateswaran et al., J. Biol. Chem. 275:14700-14707 (2000)). In addition, concurrent with this report, Repa et al., Science 289:1524-1529 (2000b) have also used T0901317 and LXRa/β−/− mice to show the requirement of LXRs in ASCA1 gene expression and the involvement of active reverse cholesterol transport as a determinant of intestinal cholesterol absorption. The present studies support an expanded role for LXRs as important regulators of bile acid synthesis, reverse cholesterol transport front peripheral tissues, intestinal cholesterol absorption, and triglyceride and phospholipid metabolism.

The LXR agonists described in this study do not activate SREBP-2 or its downstream targets. This observation is consistent with the previously noted independent regulation of SREBP-1 and -2 in hamster liver (Sheng et al., Proc. Natl. Acad. Sci. 92:935-938 (1995)). Furthermore, consistent with the observations in LXRα−/− mice (Peet et al., Cell 93:693-704 (1998)), LXR agonist treatment resulted in a reduction of cholesterol biosynthetic gene mRNA levels. Collectively, these observations provide additional support for distinct and uncoupled SREBP-1 and SREBP-2 regulation of fatty acid and cholesterol biosynthetic pathways.

In contrast to LXRα−/− mice, LXRβ−/− mice do not show any obvious lipid phenotype in response to cholesterol feeding (D. J. Mangelsdorf, unpubl.). Accordingly, it has been hypothesized that LXRα is capable of compensating for LXRβ (in LXRβ-null mice). In vitro, both LXRα and LXRβ are able to up-regulate cholesterol metabolism-associated genes such as human CETP and ABCA1 (Luo, Y. and Tall, A. R., J. Clin. Investig. 105:513-520 (2000); Costet et al., J. Biol. Chem. 275:28240-28245 (2000)). This redundancy may, at least in part, explain compensatory activity of LXRα in the LXRβ-null mouse. In contrast, LXRβ is clearly unable to compensate for the lack of LXRα (Peet et al., Cell 93:693-704 (1998)). This lack of compensation suggests that, ultimately, LXRα and LXRβ have at least some distinct primary targets in vivo. The LXR agonists described herein have been shown to be selective for LXRs over numerous other nuclear receptors. However, it should be noted that these compounds do not exhibit specificity with regard to LXRα versus LXRβ. This indiscriminant behavior may explain why normalization of the lipid phenotype associated with LXR agonist administration in this study could only be achieved by treatment of doubly deficient LXRα/β−/− mice and not with LXRα−/− mice (data not shown).

Hypertriglyceridemia results from numerous conditions including genetic defects in lipoprotein lipase and apolipoproteins CII, CIII, and E (Ghiselli et al., J. Clin. Investig. 70:474-477 (1982); Ito et al., Science 249:790-793 (1990); Parrott et al., J. Lipid Res. 33:361-367 (1992), Benlian et al., N. Engl. J. Med. 335:848-854 (1996)) and is a hallmark of a diverse range of disorders including diabetes mellitus, hypothyroidism, nephrotic syndromes, lipodystrophies (Chait, A. and Brunzell, J. D., Endocrinol. Metab. Clin. N. Am. 19:259-278 (1990)), and HIV-positive individuals undergoing treatment with protease inhibitors (Sullivan et al., AIDS 12:1393-1394 (1998)). Increasing evidence supports an independent role of hypertriglyceridemia in cardiovascular disease for both men and women in the general population and a significant clinical benefit from decreasing triglyceride levels (Hokanson et al., J. Cardiovasc. Risk 3:213-219 (1996), Brewer Jr., H. B., Am. J. Cardiol. 83:3F-12F (1999); Hodis et al., Clin. Cardiol. 22:15-20 (1999), Rubins et al., N. Engl. J Med. 341:410-418 (1999); Austin et al., Circulation 101: 2777-2782 (2000)). These observations support the need for a better understanding of the molecular determinants that control fatty acid metabolism and plasma triglyceride levels as well as the development of effective pharmacological agents that selectively reduce this risk factor. The results presented here highlight the feasibility of utilizing LXR agonists to further identify key factors that link lipogenesis to triglyceride synthesis, hepatic VLDL assembly, and secretion. Moreover, these studies demonstrate that LXRα antagonists can serve as useful therapeutics to oppose hypertriglyceridemia and reduce cardiovascular disease.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A method for modulating expression of a mammalian SREBP-1 gene, the method comprising administering a modulator compound that promotes or inhibits LXRα-mediated expression of the SREBP-1 gene to a cell that comprises an SREBP-1 gene and an LXRα polypeptide.

2. The method of claim 1, wherein the modulator compound is an agonist of LXRα and promotes LXRα -mediated expression of the SREBP-1 gene.

3. The method of claim 1, wherein the modulator compound promotes or inhibits LXRα-mediated expression of the SREBP-1c transcript.

4. The method of claim 2, wherein the modulator compound is 24,25-epoxycholesterol.

5. The method of claim 1, wherein the modulator compound is an antagonist of LXRα and inhibits LXRα-mediated expression of the SREBP-1 gene.

6. The method of claim 5, wherein the cell further comprises one or more genes that encode an enzyme involved in fatty acid and triglyceride metabolism and contacting the cell with the modulator compound inhibits expression of one or more of the genes that encode an enzyme involved in fatty acid and triglyceride metabolism.

7. The method of claim 1, wherein the enzyme involved in fatty acid and triglyceride metabolism is selected from the group consisting of fatty acid synthase, acetyl CoA carboxylase, steroyl CoA desaturase, and lipoprotein lipase.

8. The method of claim 1, wherein the cell is in a mammal.

9. The method of claim 8, wherein the mammal is a human.

10. The method of claim 8, wherein the modulator compound is an antagonist of LXRα and triglyceride levels in the mammal are reduced.

11. The method of claim 8, wherein the modulator compound is an antagonist of LXRα and insulin levels in the mammal are reduced.

12. A method of modulating triglyceride levels in a mammal, the method comprising administering to the mammal an effective amount of a modulator compound that inhibits LXRα-mediated expression of an SREBP-1 gene in cells of the mammal.

13. The method of claim 12, wherein mammal is a human.

14. A method of prescreening to identify a candidate therapeutic agent that modulates SREBP-1 expression in a mammal, the method comprising:
providing a reaction mixture which comprises:
a polypeptide that comprises an LXRα ligand binding domain (LBD);
a ligand for LXRα; and
a test compound; and
determining whether the amount of LXRα ligand that binds to the LBD is increased or decreased in the presence of the test compound relative to the amount of ligand that binds to the LBD in the absence of the test compound;
wherein a test compound that causes an increase or decrease in the amount of LXRα ligand binding to the LBD is a candidate therapeutic agent for modulation of SREBP-1 expression in a mammal.

15. The method of claim 15, wherein the method further comprises administering the candidate therapeutic agent to a cell which comprises a SREBP-1 gene to determine whether the candidate therapeutic agent modulates expression of the SREBP-1 gene in the cell, and/or expression of a gene that is regulated by SREBP-1.

16. The method of claim 15, wherein the gene that is regulated by SREBP-1 encodes an enzyme involved in fatty acid and/or triglyceride metabolism.

17. The method of claim 16, wherein the enzyme involved in fatty acid and/or triglyceride metabolism is selected from the group consisting of fatty acid synthase, acetyl CoA carboxylase, steroyl CoA desaturase, and lipoprotein lipase.

18. The method of claim 15, wherein the gene that is regulated by SREBP-1 encodes an enzyme involved in adipocyte differentiation.

19. The method of claim 15, wherein the cell is in a mammal.

20. The method of claim 14, wherein the ligand for LXRα is a peptide sensor.

21. The method of claim 20, wherein the peptide sensor is derived from an RXR.

22. The method of claim 20, wherein the peptide sensor is derived from a coactivator or corepressor.

23. The method of claim 22, wherein the coactivator is SRC-1 or NCOR.

24. The method of claim 20, wherein the peptide sensor is derived from a coactivator and comprises an amino acid sequence LXXLL, where X is any amino acid.

25. The method of claim 20, wherein the peptide sensor is derived from a corepressor and comprises an amino acid sequence IXXII, where X is any amino acid.

26. The method of claim 20, wherein the peptide sensor comprises a detectable label.

27. The method of claim 14, wherein the ligand for LXRα is a coactivator or corepressor.

28. The method of claim 14, wherein the ligand for LXRα is an oxysterol.

29. The method of claim 28, wherein the oxysterol is 24,25-epoxycholesterol.

30. The method of claim 14, wherein the amount of binding is determined using a FRET assay.

31. The method of claim 14, wherein the amount of binding is determined using a fluorescence polarization assay.

32. The method of claim 14, wherein the amount of binding is determined using ELISA.

33. The method of claim 14, wherein the amount of binding is determined using a direct binding assay.

34. A method for ameliorating a condition associated with abnormal SREBP-1 expression in a mammal, the method comprising administering to the mammal a therapeutically effective amount of a LXRα antagonist.

35. The method of claim 34, wherein the condition associated with abnormal SREBP-1 expression is hypertriglyceridemia.

36. The method of claim 34, wherein the condition associated with abnormal SREBP-1 expression is lipodystrophy.

37. The method of claim 36, wherein the lipodystrophy is congenital generalized lipodystrophy.

38. The method of claim 34, wherein the condition associated with abnormal SREBP-1 expression is insulin resistance.

39. The method of claim 34, wherein the condition associated with abnormal SREBP-1 expression is an elevated plasma insulin level.

40. The method of claim 34, wherein the condition associated with abnormal SREBP-b 1 expression is hyperglycemia and/or diabetes mellitus.

41. The method of claim 34, wherein the condition associated with abnormal SREBP-1 expression is a syndrome associated with treatment of AIDS by administration of an HIV protease inhibitor, which syndrome is characterized by one or more of lipodystrophy, insulin resistance and hyperlipidemia.
